(19) United States
(12) Patent Application Publication
(10) Pub. No.: US 2011/0092488 A1
(43) Pub. Date: Apr. 21, 2011

(54) Quninoline Methanol Compounds for the Treatment and Prevention of Parasitic Infections

(76) Inventors: Geoffrey Dow, Washington, DC (US); Tiffany Heady, Washington, DC (US); Kirsten Smith, Silver Spring, MD (US)

(21) Appl. No.: 12/084,157

(22) PCT Filed: Oct. 27, 2006
(86) PCT No.: PCT/US2006/042047

§ 371 Date: Dec. 30, 2010

Related U.S. Application Data
(60) Provisional application No. 60/731,985, filed on Oct. 28, 2005.

(51) Int. Cl. A61K 31/55; A61K 31/4709; A61K 31/47; C07D 215/14; C07D 401/06; A61P 33/00; A61P 33/06; A61P 33/02; A61P 31/06

(52) U.S. Cl. 514/217.07; 514/314; 514/311; 546/176; 546/173; 540/597; 540/481

(57) ABSTRACT

Malaria is responsible for 1-2 million deaths and 300-500 million clinical cases annually and is an ever present problem for the military, tourists and business travelers. Mefloquine is known and used for malaria prophylaxis. However it is associated with neurological effects. The present invention is directed to providing new and novel quinoline analogs that are less neurotoxic than mefloquine without compromising efficacy. The present invention is also directed to the prevention and treatment of other microbial, parasitic, protozoan, bacterial and fungal diseases.

This application claims priority from U.S. provisional application Ser. No. 60/731,985, filed Oct. 28, 2005.

### BACKGROUND OF THE INVENTION

In the late 1960s to early 1970s, Plasmodium falciparum malaria in South East Asia began to develop resistance to all of the available antimalarial drugs. Cure rates were 11-20% and 26-50% for chloroquine and quinine, respectively, and had declined to only 90% for the triple combination of quinine/pyrimethamine/dapsone. All of these regimens were associated with adverse side effects. As a consequence, the U.S. Army began routinely employing two experimental antimalarial drugs, WR030090 and WR033063, for the treatment of recrudescent malaria infections at the Walter Reed Army Medical Center. Subsequent field trials demonstrated that WR030090, a quinolinyl methanol, exhibited cure rates of at least 88% and was better tolerated than quinine.

Shortly thereafter, mefloquine was discovered and was developed commercially by Hoffman La Roche and the U.S. Army. Mefloquine exhibited a long half-life in humans, and this desirable property facilitated its administration as a single dose for malaria treatment and as a once weekly dosing for prophylaxis. In contrast, WR030090 was only partially effective as a prophylactic agent, required a similar dosing regimen as quinine to effect cures, and was subsequently abandoned. However, it is important to recognize that this occurred because of unfavorable pharmacokinetic characteristics, not as a consequence of unacceptable toxicity. Mefloquine combined with artesunate remains one of the most effective combination agents for treatment of malaria. Mefloquine is also the only once-weekly drug approved for malaria chemoprophylaxis in the United States that, barring the That border regions, is effective in almost all areas of the world. Mefloquine is also useful as an antimalarial, antimicrobial, antiparasitic, antiprotozoan, antibacterial and an antifungal agent. Furthermore, mefloquine is also being explored for central nervous system disorders including Parkinson's and prion diseases.

However, mefloquine use has been hampered for several reasons. Firstly, mefloquine is relatively expensive compared to other antimalarials, which limits its accessibility to developing countries. More importantly, mefloquine use is associated with debilitating neurological effects, and other milder, but nevertheless concerning effects including ataxia, dizziness, vertigo, insomnia and anxiety. These negative characteristics have limited the scope of the possible clinical utility of the drug.

Numerous quinolinyl methanols related to mefloquine exhibit phototoxicity. This must be appropriately considered in the context of design of next generation analogs. Phototoxicity is thought to result from pi-bond conjugation of the quinoline and any attached ring systems. Other investigators have partially resolved this issue by addition of bulky atoms (bromine, tert-butyl etc) to the ortho and 3 positions of the phenyl and quinoline rings. Steric effects then prevent alignment and conjugation of the ring systems.

It is for these reasons that the present invention, as discussed below, provides a class of compounds, methods of use and methods of making compounds derived from modification of the mefloquine skeleton that result in a more useful pharmacological agent for the prevention and treatment of malaria, and other microbial, parasitic, protozoan, bacterial and fungal diseases by improving activity and neurological therapeutic indices.

### SUMMARY OF THE INVENTION

It is, therefore an objective of the present invention to provide a class of compounds that are less neurotoxic than mefloquine.

It is also another objective of the present invention to provide a class of compounds that are at least as efficacious as mefloquine.

It is also another objective of the present invention to provide a class of compounds that can be used as antimalarials, antimicrobials, antiparasitics, antiprotozoans, antibacterials and antifungals.

It is also another objective of the present invention to provide a means of making compounds that are less neurotoxic than mefloquine as well as being efficacious as antimalarials, antimicrobials, antiparasitics, antiprotozoans, antibacterials and antifungals. These and other objectives are discussed below.

### DESCRIPTION OF THE FIGURES

FIG. 1 shows neurotoxicity of mefloquine and the mefloquine metabolite.

FIG. 2(a) shows that WR069878 is less neurotoxic than mefloquine.

FIG. 2(b) shows that WR069878 does not disrupt calcium homeostasis in the same manner as mefloquine.

FIG. 2(c) shows that the neurotoxicity of WR069878 is not blocked by reversal agents that do have such a mitigating effect on mefloquine-induced neurotoxicity.

FIG. 3 shows phototoxicity pharmacophore maps.

FIG. 4 is an isobologram showing that the combined effect of a particular AAQM, WR007524, and azithromycin, against Plasmodium falciparum W2.

FIG. 5 is an isobologram showing that the combined effect of a particular AAQM, WR007524, and azithromycin, against Plasmodium falciparum D6.

FIG. 6 is an isobologram showing that the combined effect of a particular AAQM, WR007524, and azithromycin, against Plasmodium falciparum TM91C235.

### DETAILED DESCRIPTION OF THE EMBODIMENTS

The present invention is directed to classes of compounds that are capable of providing efficacy against malaria, and parasitic, protozoan, bacterial and fungal infections and diseases. These compounds may be utilized as preventative measures against or as treatment for malaria and other microbial diseases and infections.

The compounds of the present invention alleviate neurotoxicity and improve activity, whilst retaining the desirable properties of a practical and useful pharmacological agent. These principles are broadly applicable to the treatment and prevention of any of the conditions including infectious disease and immune disease against which mefloquine can be applied. Thus, the present invention provides mefloquine analog compounds and methods for identifying and making these less neurotoxic mefloquine analogs that also retain the properties of useful drug substances for treatment of a variety of diseases and conditions. The compounds of the present invention can be administered orally, topically, transdermally and parenterally.

In a preferred embodiment, the present invention is directed towards quinoline methanol compounds related to WR030090. These compounds differ from mefloquine in that the piperidine ring is replaced by an N-alkyl functionality at the 4 position, the trifluoromethyl group at the 2 position is substituted with an aryl grouping and the trifluoromethyl group at the 8 position is replaced with various combinations of H or halogens at the 6, 7 and 8 positions of the quinoline rings. As shown in (I) below, alkylaminoquinolinyl compounds are effective antimalarial agents in vitro and in vivo. The antimalarial activity of these compounds was evaluated as described in Materials and Methods, below.

Table 1, below, provides biological data for alkyl amino quinoline methanol compounds (hereinafter referred to as AAQMs) that are starting compounds for the new and novel compounds as taught in the present invention. These compounds were chosen because they exhibited much greater activity than mefloquine against P. falciparum, in vitro. The preferred compound of the present invention, WR069878, showed the greatest efficacy by exhibiting IC90s against TM90C2A, TM91C235, D6 and W2 of 16, 11.7, 5.3 and 0.49 ng/ml, respectively. Comparable values for mefloquine were 101, 89, 20 and 3.9 ng/ml.

Also, as shown in Table 1, below, with respect to in vivo activity, the majority of the AAQMs exhibited some curative effects in the P. berghei-mouse model when administered subcutaneously. With the exception of WR177973, all were substantially more effective by the oral route, with minimum curative doses ranging from <1.25-10 mg/kg/day for 3 days for most analogs.

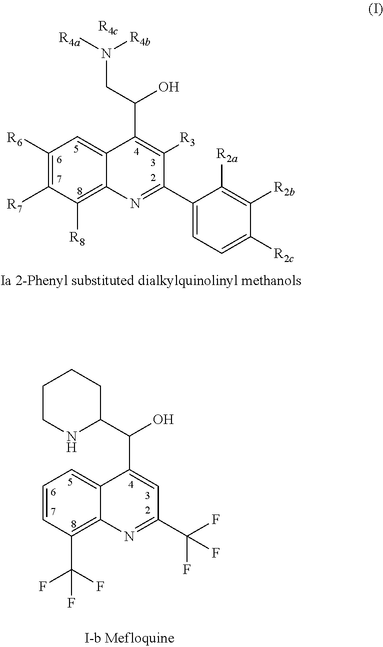

Where:

R₂ₐ is H or t-butyl; R_{2b} is H or Cl; R_{2c} is H, Cl, or F; R₃ is H; R₄ₐ is H, ethyl, butyl or hexyl; R_{4b} is H, ethyl, butyl, or hexyl; R_{4c} represents an addition to the N or the amino side chain; R₆ is H, methyl or Cl; R₇ is H, F, or Cl; and R₈ is H, methyl or Cl.

TABLE 1

| | | | | | | | | | | | Predicted | | Activity against | | |
|---|---|---|---|---|---|---|---|---|---|---|---|---|---|---|---|
| | | | | | | | | | | | solubility | | P. falciparum | Neurotoxicity | |
| | | | | | | | | | | | pH 5 5 | Log | TM91C235 | ranking | |
| Compound | 2a* | 2b* | 2c* | 3* | 4a* | 4b* | 4c* | 6* | 7* | 8* | (mg/ml) [symbol] | P [symbol] | (IC₅₀ in ng/ml) | (IC₅₀ in μM) | [symbol] |
| Mefloquine | CF [symbol] | | | H | Piperidine [symbol] | | | CF [symbol] | H | H | 8.29 | 2.87 | 15 | 20 | >12 [symbol] |
| WR030090 | H | Cl | Cl | H | Bu | Bu | H | Cl | Cl | Cl | 0.004 | 8.18 | 11 | 2000 | >12 [symbol] |
| WR069878 | H | H | Cl | H | Bu | Bu | H | Me | Cl | H | 0.019 | 8.04 | 1.5 | 200 | 38 |
| WR176399 | H | H | Cl | H | Bu | H | H | Me | H | Me | 1.11 | 5.47 | 0.48 | 600 | >12 [symbol] |
| WR007524 | H | H | Cl | H | Bu | H | H | Cl | H | Cl | 0.28 | 5.46 | 0.63 | 60 | >12 [symbol] |
| WR041294 | H | H | Cl | H | Et | Et | H | Cl | H | Cl | 1.59 | 5.04 | 1.1 | 60 | >12 [symbol] |
| WR081049 | H | H | Cl | H | Bu | Bu | H | H | F | H | 0.088 | 7.01 | 1.6 | 200 | 24 |
| WR035058 | H | H | Cl | H | Bu | Bu | H | Cl | H | H | 0.037 | 7.18 | 2.7 | 600 | 38 |
| WR098656 | H | H | Cl | H | Bu | Bu | O⁴ | Cl | H | Cl | 19.0 | 4.98 | 3.3 | 60 | >12 [symbol] |
| WR074086 | H | H | F | H | Bu | Bu | H | Cl | H | Cl | 0.043 | 7.10 | 3.4 | 2000 | 46 |
| WR211925 | H | H | Cl | H | Bu | Bu | H | H | H | Me | 0.090 | 7.29 | 5.2 | 600 | 54 |
| WR029252 | H | H | Cl | H | Bu | Bu | H | Cl | H | Cl | 0.004 | 7.74 | 7.6 | 600 | >12 [symbol] |
| WR211679 | H | H | H | H | Hex | Hex | H | H | H | Cl | 0.024 | 8.63 | 7.7 | 2000 | 64 |
| WR177973 | TBᵤ^{b} | | | H | Bu | Bu | H | Cl | H | H | 0.43 | 6.78 | 7.9 | 200 | 15 |
| WR106752 | H | H | I | H | Bu | Bu | H | Cl | H | Cl | 0.029 | 8.17 | 8.0 | 2000 | NT |

Where:
*Denotes position on quinoline ring at which substitution occurs, as in (I). Groups 2a-c denote either substitution at the 2-position or modifications to the phenyl ring attached to the quinoline ring at the 2 position. Groups 4a-c denote modifications to the N side chain at the 4 position;
^{a}Trifluromethyl group is attached at 2-position of the quinoline ring;
^{b}t-Butyl group is attached to the 2-position of the quinoline ring;
^{c}Piperdine ring as in mefloquine in Ib.
^{d}N-oxide;
^{e}Calculated using ACD/LogD Sol Suite;
^{f}Minimum daily dose that cured at least one of five P. berghei-challenged mice. Drugs were given s.c. for three days;
^{g}Minimum daily dose that cured at least one of five P. berghei-challenged mice. Drugs were given orally for three days. Compounds were tested at University of Miami unless otherwise indicated. Greater/less than symbols indicate that the MED was outside the dose range tested as indicated;
^{h}These compounds were tested at AFRIMS, Thailand;
ⁱClinical outcome in groups of two P. falciparum-challenged Aotus. Cure means that both monkeys were parasite free 90 days after treatment. Failure means that monkeys were rescued with mefloquine. R means that infections were cleared, and the number indicates the average day of recrudescence for both monkeys; and
NT = Not tested due to insolubility or insufficient compound. [symbol]
indicates data missing or illegible when filed

Table 2, below, provides a relationship between efficacy of selected AAQMs in Aotus monkeys, in vitro antimalarial activity and plasma concentrations. Selected analogs were then tested for efficacy against P. falciparum in Aotus monkeys. Cures were observed with WR069878 and WR035058. The testing off WR069878 was expanded, and a summary of the data are presented in Table 3. These data indicate that WR069878 cures P. vivax infections oral in a three day regimen. WR074086 and WR176399 cleared parasitemias, but recrudescence was subsequently observed. Clinical failure was associated with high in vitro IC90 (> or = to 20 ng/ml) against P. falciparum TM91C235 and/or relatively low plasma concentrations, as shown in Table 2, below. These issues are relevant in the context of instructions for the creation of improved next generation quinoline methanols discussed later in this invention.

This data shows that a number of AAQMs with structures related to WR069878 are more potent, in vitro, than mefloquine. They are thus more effective against microorganisms that are mefloquine resistant (e.g. against P. falciparum TM90C2A). Some of them, in particular WR069878 and WR035058, have excellent oral efficacy without toxicity in vivo.

TABLE 2

| | | | Plasma conc | | Plasma conc. | | Mean peak | Mean |
|---|---|---|---|---|---|---|---|---|
| | Outcome in | TM91C235 | on day 1 (ng/ml) | | on day 7 (ng/ml) | | concentration | peak |
| Compound | Aotus | IC90 (ng/ml) | 2 h | 24 h | 2 h | 24 h | (ng/ml) | conc/IC90 |
| WR035058 | Cure | 8.5 | 454 | 84 | 371 | 105 | 413 | 49 |
| WR069878 | Cure | 3.3 | 38 | 16 | 91 | 40 | 65 | 20 |
| WR074086 | Clear (R25) | 7.0 | 179 | 4.0 | 78 | 78 | 129 | 18 |
| WR176399 | Clear (R21) | <0.5 | 27 | 2.7 | 30 | 3.8 | 28 | 49 |
| WR029252 | Failure | 20 | 67 | 1.0 | 21 | 1.0 | 44 | 2.2 |
| WR030090 | Failure | 52 | 597 | 11 | 16 | 5.6 | 307 | 5.9 |

TABLE 3

| Dose | Number | Type of | | |
|---|---|---|---|---|
| (mg/kg/day) | of Days | treatment^{a} | Species^{b} | Outcome^{c} |
| 20 | 1 | Retreatmcnt | P. vivax-AMRU1 | Cure |
| 20 | 1 | Retreatment | P vivax-AMRU1 | Clear (8) |
| 80 | 1 | Retreatment | P vivax-AMRU1 | Clear (11) |
| 80 | 1 | Retreatment | P. vivax-AMRU1 | Cure |
| 0.625 | 3 | Primary | P. vivax-AMRU1 | Failure |
| 0.625 | 3 | Primary | P. vivax-AMRU1 | Failure |
| 2.5 | 3 | Primary | P. vivax-AMRU1 | Failure |
| 2.5 | 3 | Primary | P vivax-AMRU1 | Failure |
| 10 | 3 | Primary | P. vivax-AMRU1 | Clear (7) |
| 10 | 3 | Primary | P. vivax-AMRU1 | Cure |
| 40 | 3 | Retreatment | P. vivax-AMRU1 | Cure |
| 40 | 3 | Retreatment | P. vivax-AMRU1 | Cure |
| 10 | 7 | Primary | P. falciparum FVO | Cure |
| 10 | 7 | Primary | P. falciparum FVO | Cure |

Where:
^{a}shows primary treatment and retreatments. Primary treatment refers to the initial treatment given when parasitemia reached 5000 parasites/μL. Retreatment refers to the administration of an additional course of treatment in the event of recrudescence after, or failure of, the primary treatment.
^{b}denotes strains/species that are chloroquine-resistant.
^{c}shows the various possible outcomes. When the outcome of treatment was clearance, the number in brackets indicates the number of days before parasites recrudesced.

In accordance with the present invention, and as shown in Table 4, below, these characteristics are correlated with ‘opening’ of the piperidine ring of mefloquine, since ‘open-chain’ N,N-dialkylaminoquinolines display greater activity compared to 4-quinoline carbinolamines (4QCs) in which the 4-amino side chain comprises a piperidine ring (as in mefloquine, structure 1b). Furthermore, as shown in Table 4, this same structural change renders AAQMs less neurotoxic than 4QCs. Thus, quinoline methanols that do not possess a piperidine side chain are intrinsically more active than those that do not and such structural modification is, therefore, imperative for improving the activity and therapeutic index of the mefloquine scaffold against a number of conditions.

TABLE 4

| Parameter | Mefloquine | AAQMs* | 4-QCs** |
|---|---|---|---|
| Median IC50 against P. falciparum | 35 | 17 | 26 |
| TM91C235 (nM) | | | |
| Median IC50 against rat | 20 | 600 | 33 |
| neurons (μM) | | | |
| Therapeutic index (*1000) | 0.57 | 35 | 1.3 |
| Therapeutic index relative | 1.0 | 61 | 2.3 |
| to mefloquine | | | |

*Data from AAQMS present in Table 1
**A 4-QC is a mefloquine analog that possesses a 4 aminoalcohol side chain comprising a piperidine ring (as in Structure 1b for mefloquine). These data are for a group of 4Qcs, containing a phenyl grouping at the two position.

These observations are important when one considers the in vivo neurotoxicity of mefloquine. Mefloquine has been shown to induce neurodegeneration of brain stem nuclei in rats given pharmacologically relevant doses of the drug. This is associated with neurological signs (ataxia) similar to the clinical neurological effects (ataxia/dizziness/vertigo) of mefloquine. The lesions induced by mefloquine are consistent with a neurocytotoxic effect of the drug in vivo. Previous studies demonstrate that the mechanism of neurocutotoxicity of mefloquine is via disruption of calcium homeostasis.

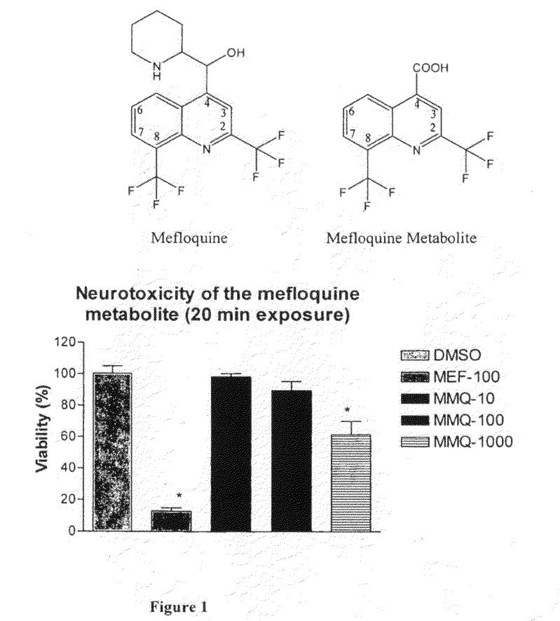

The importance of removal of the piperidine ring for reducing neurotoxicity is underscored by comparison of the neurotoxicity of mefloquine with its metabolite. The neurotoxicity of mefloquine and its principal in vivo metabolite were evaluated as described in Materials and Methods, below. The mefloquine metabolite lacks the piperidine ring of mefloquine. As shown in FIG. 1, mefloquine is at least an order of magnitude more neurotoxic than its metabolite (100% killing at 100 micromolar versus only partial killing at 1000 microM). The metabolite exhibits almost an order of magnitude less neurotoxicity than mefloquine. Mefloquine is almost 100% lethal at a concentration of 100 micromolar, but the mefloquine metabolite is only partially lethal at a concentration of 1 millimolar. Difference is due to the piperidine ring of mefloquine.

Therefore in accordance with our present invention, it is the piperidine ring that governs the degree and mechanism of neurotoxic effects observed by mefloquine, and that this can be mitigated in an anti-infective agent.

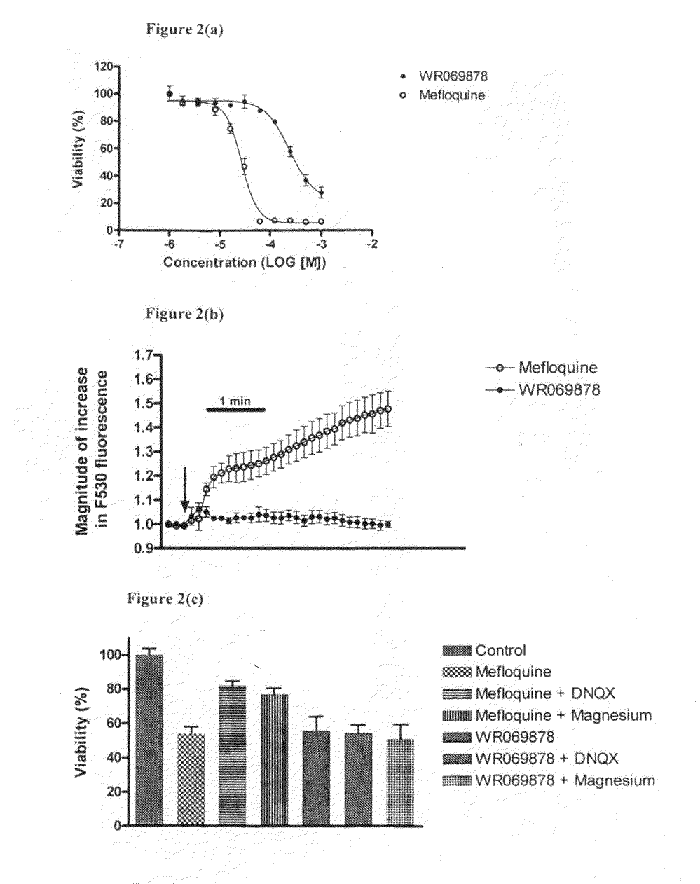

Also, in accordance with the present invention, the neurological effects of WR069878, as shown in FIGS. 2(a), 2(b), and 2(c) provide that WR069878 is significantly less neurotoxic than mefloquine. FIG. 2(a) shows that mefloquine, with an IC₅₀ of 27 μM, is more neurotoxic than WR069878 with an IC₅₀ of 242 μM. This effect does not result from a difference in solubility, since both drugs dissolved without evidence of precipitation across the concentration range tested. The mechanism of neurotoxicity of mefloquine also appears to be different, since WR069878 did not disrupt calcium homeostasis to the same extent as mefloquine, as shown in FIG. 2(b). Mefloquine, but not WR069878 at a concentration of 100 μM increases intracellular calcium concentrations, as indicated by the relative increase in Fuo3 fluorescence, as measured by confocal microscopy. The disruption of neuronal calcium caused by mefloquine occurs as a consequence of discharge of ER calcium store and influx of extracellular calcium through unknown mechanisms. Drugs were added at the time indicated by the arrow.

Additionally, as shown in FIG. 2(c), the neurotoxicity of WR069878 is not inhibited by 6,7-dinitroquinoxaline-2,3-dione (DNQX) and supra-physiological magnesium, agents shown to partially protect neurons from mefloquine-induced neurotoxicity. This is because the neurotoxicity of mefloquine, but not WR069878, is blocked by DNQX and supra-physiological magnesium, indicating that the compounds have different mechanisms of toxicity. Bars represent standard errors in all cases. In this context, WR069878 represents an example of a quinoline methanol in which the therapeutic index has been improved relative to mefloquine by elimination ‘opening’ of the piperidine ring.

In accordance with the present invention, Table 4, and FIGS. 1, 2(a), 2(b) and 2(c), collectively show that mefloquine analogs without a piperidine ring are less intrinsically neurotoxic that those with a piperidine ring, exhibit a different mechanism of toxicity and have greater therapeutic indices than mefloquine or mefloquine analogs containing a piperidine ring. Thereby, the present invention provides a method by which novel and commercially viable mefloquine analogs that are designed to be used to prevent and treat a variety of microbial infections and diseases, for which neurologic therapeutic index is improved. However, in accordance with the present invention, other factors must be considered in the design of mefloquine analogs. These factors include (i) maintenance of the intrinsic metabolic stability imparted by the piperidine ring in a new and novel series of analogs, (ii) incorporation of structural motifs that mitigate phototoxicity while improving activity, (iii) incorporation of structural motifs that reduce the lipophilicity of the scaffold to facilitate better absorption, and (iv) incorporation of structural motifs that increase the polar surface area, increase the acidity and lower the LogP in such a manner that passage across the blood-brain-barrier and PgP substrate affinity. The latter changes result in mefloquine analogs with a further improved neurologic therapeutic index.

In accordance with a preferred embodiment of the present invention, the choice of non-piperidine side chain substituent must be carefully chosen to balance metabolic stability and enhanced potency with reduced neurotoxicity. As discussed above, the removal of the piperidine ring of mefloquine results in improved activity and a higher therapeutic index. However the choice of replacement substituent for the side chain must be carefully considered, since the 4 side chain has an effect on other properties of the molecule besides neurotoxicity. Alkylaminoquinoline compounds (AAQMs) showing relatively potent in vitro antimalarial activity and neurotoxicity contained either short alkyl amino chains, such as WR041294 versus WR029252 as shown in Table 1, or had one chain removed such as WR007524 versus WR029252, also as shown in Table 1.

The metabolic stability of several of the AAQMs have been determined as described in the materials and methods, below.

As shown in Table 1, many of the singly alkylated analogs (secondary amines) such as WR176399 and WR007524 showed metabolic stability similar to that associated with mefloquine. In contrast, many of the dialkylated analogs, such as WR069878, were less metabolically stable, also as shown in Table 1. The N,N-dialkyl analogs were metabolized in all species primarily (>90%) by N-dealkylation, yielding the corresponding secondary amine metabolite, as in WR069878, as shown in the synthesis pathway (II), below.

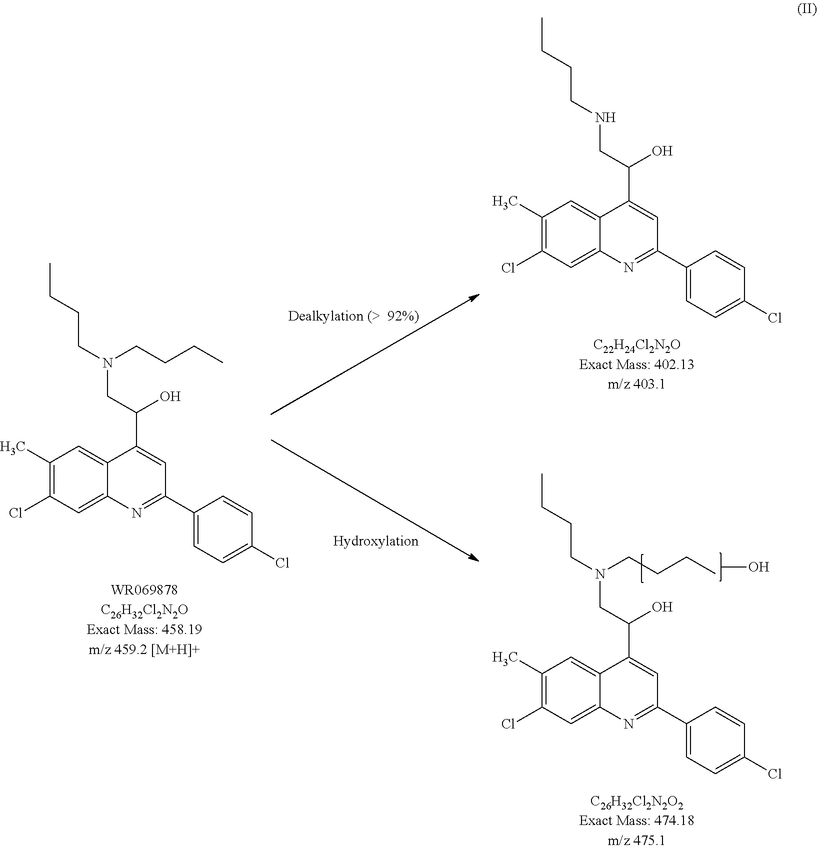

Hydroxylation is a minor but secondary route of metabolic transformation. Singly alkylated analogs such as WR176999 are metabolized to a much lesser extent via hydroxylation, as with, WR176399, and/or N-dealkylation as with WR041294, or not at all, as with WR007524. Metabolism was similar across all the species tested (rat, human, mouse, and rhesus).

As shown in (III)(a) through (d) below, and in a preferred embodiment of the present invention, a 4 amino side chain that is resistant to N-dealkylation must be selected so as to achieve an appropriate balance between neurotoxicity and metabolic stability. In another preferred embodiment of the present invention, these properties must be appropriately balanced. One approach, as discussed above, would be to utilize an N-butyl side chain as in (III) (a) through (d). Alternatively the piperidine side chain can be replaced with an alternative N-containing ring, as in (III) (b) through (d). Alternatively, one could use a dialkyl or other substituted compound as pro-drugs for more metabolically stable and active compounds. Other modifications that result in slower abstraction at positions alpha to the amine nitrogen are also within the scope of the present invention.

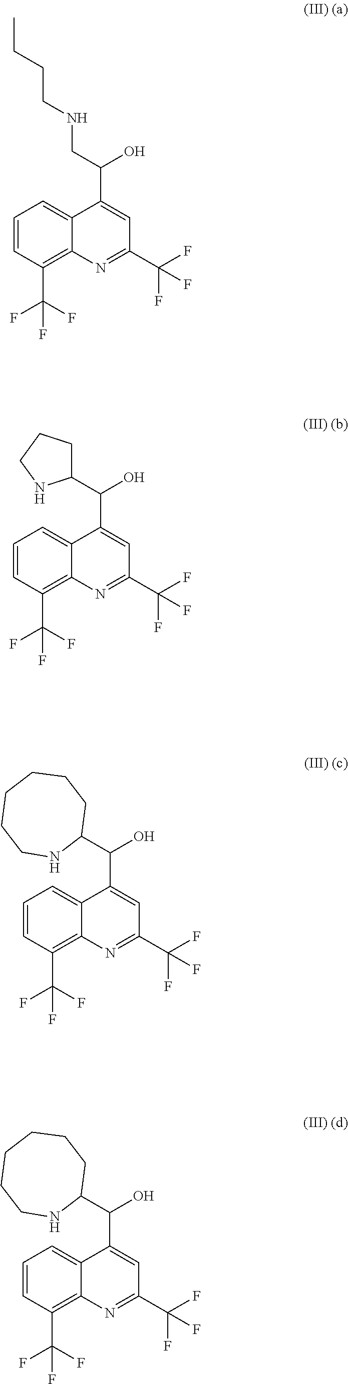

As shown in (IV)(a) through (g) of a preferred embodiment of the present invention, careful consideration must be given to the choice of substituent at the 2 position of the quinoline ring. This is because the choice of substituent can both improve activity and impart phototoxicity and shown in the structures (IV) (a) through (g), below. Antimalarial activity is improved by substitution of the trifluoromethyl in IV (c) and (d) for a phenyl group in IV (a) and (b) at the 2 position.

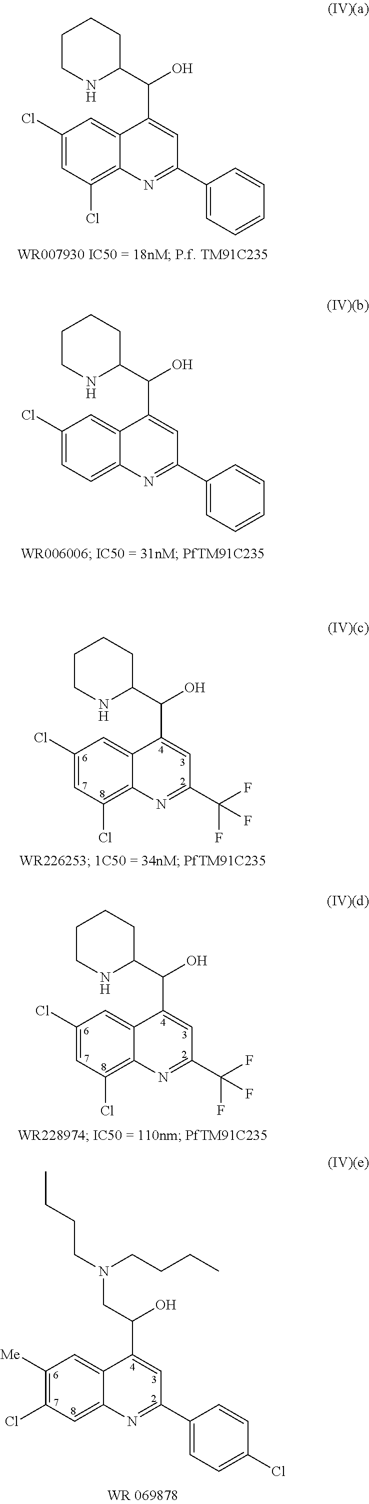

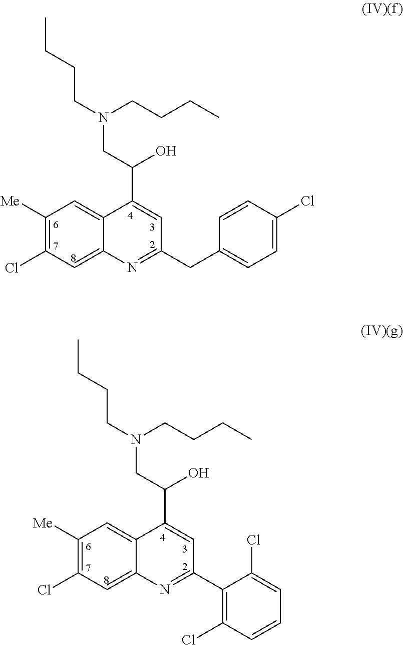

Historically, this functionality was incorporated into this set of analogs as a blocking group for maintaining metabolic stability. However, the disadvantage of the phenyl substituent at the 2 position of the quinoline ring is that it is associated with phototoxicity. We confirmed this using three different techniques to evaluate phototoxicity for three quinoline methanols, WR007930, WR030090 and mefloquine, for which there is clinical precedent. As outlined in Table 5, below, WR007930 was abandoned during clinical development due to phototoxicity. Mild phototoxicity was observed after WR030090 administration in some patients. Mefloquine is not phototoxic. The structures of mefloquine and WR030090 are illustrated in I and Table 1, and the structure of WR007930 is shown in (IV)(a). WR030090 or WR007930 but not mefloquine contains a 2-position phenyl group.

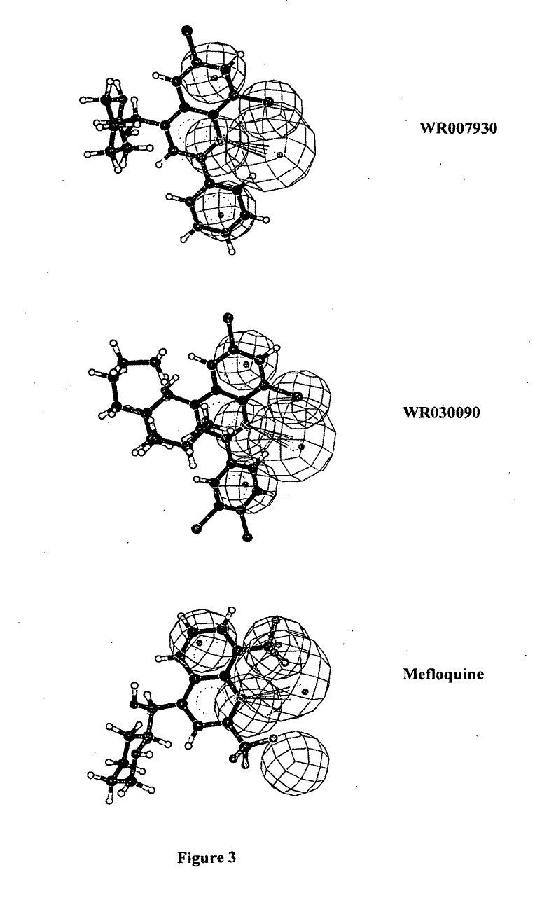

Using the first method, we compared data from historical experiments in mice. Both WR030090 and WR007930 but not mefloquine were phototoxic, as outlined in Table 5, below. This was confirmed in an in vitro 3T3 NRU assay by us, as described in Materials and Methods. Again, as outlined in Table 5, mefloquine, the only non-phenyl-containing compound, is not photo-toxic, as evidenced by a lower photo-irritancy factor (<5). Further evidence of involvement of the phenyl grouping was revealed after we developed a phototoxicity pharmacophore, based on published data for a yeast model as per Ison, et al.; Phototoxicity of Quinoline methanols and other drugs in mice and yeast; J. Invest. Dermatol. (52)193-198 (1969), which is incorporated herein, by reference. Features required for phototoxicity include a hydrogen bond acceptor and an aliphatic hydrophobic and two aromatic hydrophobic functionalities, as shown in FIG. 3. The phototoxicity pharmacophore was generated based on published studies (Ison and Davis, 1969), reporting the minimum phototoxic concentrations of various quinoline methanols in an in vitro yeast growth inhibition assay. All the features of the pharmacophore map to WR007930 and WR030090. However, the aromatic hydrophobic functionality associated with the 2-position phenyl group does not map to mefloquine. Based on the pharmacophore mapping, the estimated minimum phototoxic concentrations for WR030090 and WR007930 is 110 and 170 mg/ml, respectively, as shown in Table 5. In the case of mefloquine, as indicated in FIG. 3, the 2 position trifluoromethyl did not map to the pharmacophore, and the estimated minimum phototoxic concentration was correspondingly higher, as in Table 5, below. These data indicate the potential for phototoxicity exists across a number of biological systems if a 2-position phenyl group is present. This is likely due to nuclear conjugation of the quinoline and phenyl rings. Mechanistically, this results in pi-electron sharing if the ring systems are coplanar.

TABLE 5

| Parameter | Clinical endpoint | Mice | Yeast | Pharmacophore | (PIF)^{e} |
|---|---|---|---|---|---|
| WR007930 | Irritation/erythema after sun | Minimum phototoxic dose is 7 | Minimum phototoxic | Estimated MPC = | 29.0 |
| | exposure (12 mg/kg po for 14 | mg/kg by ‘injection’^{c} | concentration (MFC) = | 170 mg/ml | |
| | days)^{a} | | 31 mg/ml^{c} | | |
| WR030090 | Clinically insignificant | Doses of 25 or 50 mg/kg orally | Minimum phototoxic | Estimated MPC = | 105.7 |
| | phototoxicity (10 mg/kg/day | are phototoxic^{d} | concentrations were 25 | 110 mg/ml | |
| | for 6 days in 4/124 people)^{b} | | and 500 mg/ml^{c} | | |
| Mefloquine | Not considered phototoxic | Not phototoxic at tolerated doses | Not tested | Estimated MPC = | 1.76 |
| | | | | 5600 mg/ml | |

Where:
^{a}From Pullman et al., 1946;
^{b}From Martin et al., 1973;
^{c}From Ison and Davis, 1969;
^{d}WRAIR archival data, method based on similar principles as Ison and Davis (1969); and
^{e}Photo Irritant Factor (PIF), as calculated by the 3T3 Neutral Red Uptake Phototox Prediction Software (version 2.0, developed by ZEBET); compounds with the potential to be phototoxic have a PIF > 5.0.

This leads logically to the another embodiment of the present invention which is to eliminate phototoxicity whilst maintaining antimalarial activity. Previous attempts have been hampered due to alignment of the phenyl and quinoline ring systems. To prevent this, the present invention is directed to increasing the rotational freedom via replacement of the phenyl groups with benzyl groups, or to create steric effects by addition of bulky groups to the 2 and 6 positions of the phenyl ring. These are shown as modifications to WR069878, which we know is phototoxic from the 3T3 assay (PIF>100) and the pharmacophore model (minimum photo-toxic concentration>mefloquine), as in IV(f) and IV(g).

As outlined in Table 2, poor therapeutic outcomes of some AAQMs are in part associated with relatively low plasma concentrations. Overall, plasma concentrations of the six AAQMs in Aotus monkeys were relatively low (mean peak concentrations of 28-413 ng/ml with a 10 mg/kg/day oral dose×7) compared to a single mefloquine treatment dose (peak concentrations of 800-8000 ng/ml). Lower than expected plasma concentrations for AAQMs are likely due to a combination of several contributing factors. Firstly, as outlined earlier, some of the AAQMs tested were less metabolically stable than mefloquine, which results in lower overall plasma concentrations. Secondly, most of the AAQMs were several orders of magnitude less soluble than mefloquine, as shown in Table 1. The higher solubility of mefloquine is attributable to the more polar secondary amine of the piperidine ring, whereas most of the other AAQMs contained more aliphatic tertiary amines. Finally, the predicted Log P values for most of the AAQMs were substantially higher than mefloquine, and furthermore, substantially greater than that normally observed for commercially available drugs, as shown in Table 1. Poor solubility and permeability are the two leading causes for poor drug absorption. It is therefore within the scope of the present invention to provide analogs that incorporate the alkyl and phenyl functionality, where physiochemical properties are improved by addition of more polar substituents. The polar substituents include addition of a hydroxyl group to the 4 amino side chain, as shown in a comparison between (V)(a) to (v)(b), below.

Another embodiment of this invention is directed to enhancing neurologic therapeutic indices of new and novel mefloquine analog compounds by preventing their accumulation in the central nervous system and/or interaction with blood-brain barrier P-glycoproteins (PgPs). Mefloquine accumulates in the central nervous system (CNS) relative to plasma, reaching a maximum concentration equivalent to 113 μM and 52 μM in rats and humans respectively. This level of accumulation is sufficient to allow the compound to exhibit neurological effects including disruption of calcium homeostasis and neuron killing as described by us in earlier literature. In addition, mefloquine is a PgP substrate and inhibitor. These properties are mitigatable with structural modifications that improve physiochemical properties in an appropriate manner. The rationale for this is outlined below.

TABLE 6
Physiochemical characteristics of commercial pharmaceuticals
that are not targeted to the CNS

| | | Ideal properties for mefloquine |
|---|---|---|
| | | analog with drug-like properties |
| Parameter | Mefloquine | and low CNS penetration* |
| Polar surface area | 30-47 | >70 |
| FRBs** | 3 | 9-10 |
| H Bond Acceptors | 3 | 8-10 |
| H Bond Donors | 2 | 4-5 |
| LogP*** | 2.87 | <1.5 |
| Molecular weight | 378 | 450-500 |

Where:
*Properties represent intersection of Lipinski's rules for drug-like properties and those atypical of commercial pharmaceuticals targeting the CNS. The low polar surface area (PSA), free rotatable bonds (FRBS), H Bond acceptors, H bond donors and MW and optimal LogP of mefloquine suggest it should be highly amenable to passive permeation of the CNS.
**FRBS.
***LogP is octanol:water partition coefficient and is an indicator of lipophilicity.

Mefloquine accumulation in the CNS occurs as a consequence of its ability to easily penetrate the CNS and to interact with blood-brain-barrier PgP efflux pumps. Mefloquine possesses many of the physiochemical properties that one would not select if one were designing a drug not to penetrate the CNS, as outlined in Table 6, above. This can be mitigated in new mefloquine analogs by appropriate structural modification. As shown in a comparison of (V)(c) to (V)(d). (V)(c) is an AAQM in which the piperidine ring has replaced with a dialkyl structure. This compound is preferable to mefloquine in some physiochemical respects (e.g. a greater number of FRBs) but not in others (similar or lower PSA). However addition of an acid functionality to both the alkyl side chains, as in Vd, dramatically improves most of the physiochemical properties close to, or into the desired range (see Table 7).

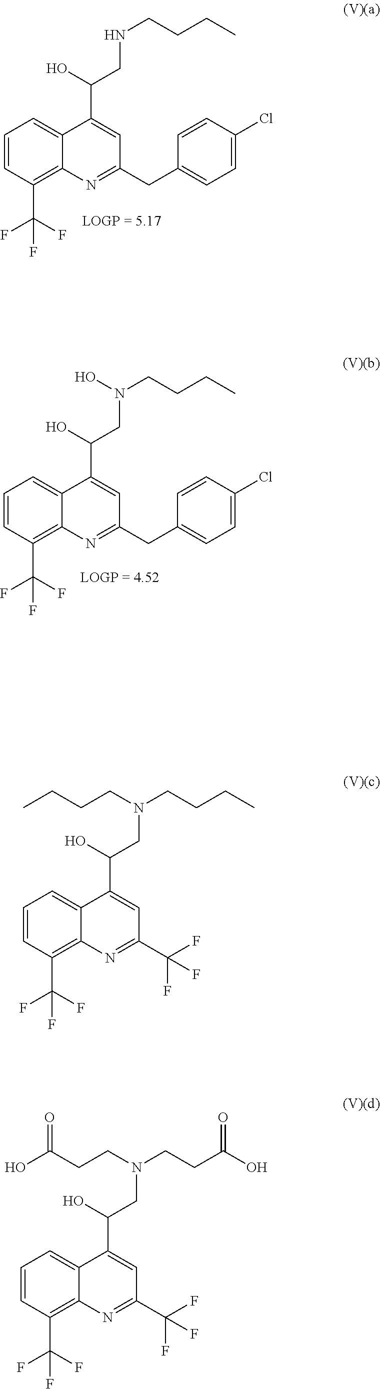

It is another embodiment of this invention that similar structural modifications (as in the change from (V)(c) to (V)(d)) also reduce the propensity of a mefloquine analog to be a PgP substrate. Mefloquine has been shown to be a substrate of human multiple drug resistance transporter (MDR1) commonly referred to as a P-glycoprotein in vitro and in vivo. This would otherwise be a desirable trait, since PgPs mediate the efflux of potentially harmful xenobiotics from the CNS. However, mefloquine also acts an inhibitor of the efflux function of PgP. This effect is mediated via its interaction with the substrate binding sites of PgP and has three important consequences. First, in comparison to an otherwise similar compound that was a substrate but not an inhibitor of PgP, mefloquine could conceivably impair its own efflux due to its inhibitory effect on PgP. Second, mefloquine impairs the efflux of other neuroactive xenobiotics. Finally, the normal PgP-mediated efflux of mefloquine from the CNS is vulnerable to impairment by other PgP inhibitors, as has been observed in mice given mefloquine with and without the PgP inhibitor elacridar. In compounds derived from natural products such, PgP substrate affinity can be reduced by increasing the acidity of the molecule. This is achieved, by addition of acidic groups to the alkyl side chains as shown in (V)(d), and outlined in Table 7, below. Here, the basic moiety of the 4 side chain is deliberately retained, since this is essential for antimalarial activity.

TABLE 7

| | | | | | H- | H- | Rule | | |
|---|---|---|---|---|---|---|---|---|---|
| Compound | LogP | MW | PSA | FRB | donors | acceptors | of 5* | pKa** | pKb*** |
| Target | <1.5 | 450-500 | >70 | 9-10 | 4-5 | 8-10 | 0 | — | — |
| Mefloquine | 2.9 | 378 | 39-47 | 3 | 2 | 3 | 0 | 12.8 | 10.0 |
| Mefloquine | 3.9 | 309 | 45-51 | 1 | 1 | 3 | 0 | 1.7 | — |
| metabolite | | | | | | | | | |
| GD208 | 2.1 | 392 | 43-53 | 4 | 1 | 1 | 0 | — | — |
| GD211 | 2.0 | 410 | 72-88 | 8 | 3 | 5 | 0 | — | — |
| GD212 | 1.52 | 406 | 61-70 | 4 | 1 | 5 | 0 | — | — |
| WR176990 | 5.8 | 436 | 30-36 | 10 | 1 | 3 | 1 | 12.5 | 8.39 |
| GD203 | 2.3 | 467 | 104-119 | 10 | 3 | 7 | 0 | 3.3 | 8.2 |

Where
*is the Number of violations of Lipinski's rules;
**denotes strongest acidic atom of 4 side chain; and
***denotes strongest basic atom of amino side chain.

It is a preferred embodiment of the present invention to provide a method of making new and novel quinoline methanol compounds that have greater activity, improved neurologic therapeutic indices, exhibit no phototoxicity, and have the requisite physiochemical properties consistent with excellent oral activity, low CNS penetrability and PgP substrate affinity. Such analogs would thus represent improvements over mefloquine and WR069878 whilst preserving the desirable feature of both. The method of making the improved quinoline analog compounds include: (i) selection of a 4-side chain substituent that appropriately balances metabolic stability, neurotoxicity and activity, (ii) selection of a 2 position substituent that optimizes antimalarial activity against phototoxicity, (iii) addition of an appropriate substitute at the 6, 7, and eight positions to optimize activity and ensure ease of synthesis (iv) introduction of additional moieties that improves oral absorption, and/or reduces the potential for blood brain barrier passage, and/or PgP substrate affinity. Each of these aspects are described below.

The piperidine ring of the 4 amino side chain is replaced with an alternative N-containing side chain. The substituent selected must be (i) resistant to N-dealkylation, (ii) must result in greater in vitro potency than mefloquine and (iii) must have reduced neurotoxicity relative to mefloquine. Specific substituent include but are not limited to, N-butyl (mono) side chain, as shown in (III)(a). Other N-alkyl or N-dialkyl structures of different types are also within the scope of the present invention. Inclusion of alternate N-containing rings in the 4-amino side chain is also within the scope of the present invention and as shown in (III)(c) through (III)(d). Additional modifications that are suitable are those that facilitate resistance to N-dealkylation by slowing hydrogen atom abstraction at positions alpha to the amine nitrogen.

The activity of mefloquine is improved by addition of a non trifluoromethyl substituent at the 2 position of the quinoline ring. This substitution does not impart phototoxicity as does the chloro-phenyl moiety of WR069878. This is facilitated via deconjugation of the phenyl and quinoline ring systems and include, but is not limited to the addition of a chloro-benzyl group to WR069878, as shown in Structure (IV)(f). This addition replaces the chloro-phenyl functionality and increases rotational freedom. Alternatively, bulky groups may be added to the 2 and 6 positions of the phenyl ring attached to the 2 position of the quinoline ring, in order to hinder alignment of the ring systems, as shown in (IV) (g).

Careful consideration must be given to the selection of substitutions at the 6, 7 and 8 positions of the quinoline ring in order to preserve activity. The trifluoromethyl group of mefloquine at the 8 position is acceptable (as in mefloquine, see (I)(b) and Table 1). This substituent would also be acceptable at the 6 or 7 positions. The combination of Me and Cl groups at the 6 and 7 positions of WR069878 is acceptable in terms of activity and could be retained (I)(a) and Table 1). Other choices of substituents, such as a combination of Cl, Me, or H would also be acceptable at the 6, 7 and 8 positions.

After reengineering the quinoline methanol scaffold as described above, one must then give careful consideration to the physiochemical properties of the resultant compound, in an effort to ensure that the molecule orally active, does not cross the blood-brain barrier to the same degree as mefloquine and/or is not a substrate for PgP. For example, the LogP and solubility of many compounds AAQMs is not necessarily desirable. In a preferred embodiment, this is alleviated by addition of more polar functional groups to either the 4 amino side chain, as shown in (V)(b) and (V)(d) or alternatively to the 6, 7 or 8 positions of the quinoline rings. Such additions include, but are not limited to, a hydroxyl, methoxy or acid groups to the 6, 7 or 8 positions. A series of structures that are new and novel quinoline compounds as taught by the present invention are shown in (VI)(a)-(c); (VII); (VIII); (IX), (X)(a)-(X)(d); (XI); (XII)(a)-(XII)(d); and (III)(a)-(XIII)(d), below.

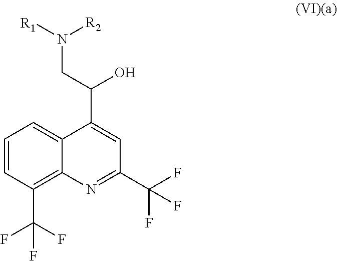

Where:

R₁ is Me and R₂ is H; R₁ and R₂ are propyl groups; R₁ is H and R₂ is a propyl group; R₁ is H and R₂ is CH₂CHOH—CH₂—CH₃; R₁ is H and R₂ is CH₂—CH₂—CHOH—CH₃; R₁ is H, R₂ is CH₂—CH₂—CH₂—CH₂OH; R₁ is OH and R₂ is butyl; R₁ is a butyl group and R₂ is CH₂OH; R₁ is butyl and R₂ is CH₂—CH₂—COOH; R₁ is CH₂—CH₂—COOH and R₂ is CH₂—CH₂—COOH; R₁ is H and R₂ is CH₂—CH₂—COOH; or R₁ and R₂ are cyclopropyls.

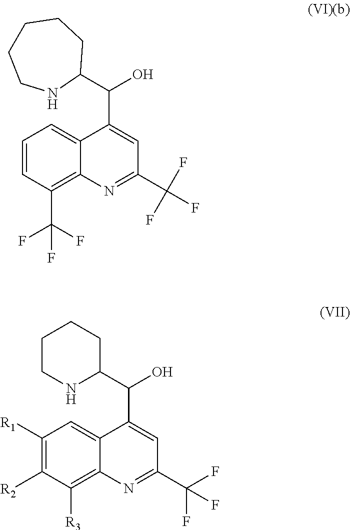

Where R₁ is Me, R₂ is Cl and R₃ is H; R₁ is Cl, R₂ is Me, R₃ is H; R₁ and R₂ are Me, R₃ is H; R₁ and R₂ are Cl and R₃ is H; R₁ is H, R₂ and R₃ are Cl; R₁ and R₂ are H and R₃ is a hydroxy group; R₁ and R₂ are H and R₃ is CH₂OH; R₁ and R₂ are H and R₃ is ethanone; R₁ and R₂ are H and R₃ is methylhydroxy; or R₁ and R₂ are H and R₃ is trifluoromethoxy.

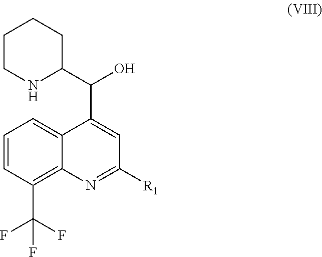

Where R₁ is 2,6-dichlorophenyl; 2,6-dimethylphenyl; 2-6-bis(trifluoromethyl)phenyl; 4-chlorobenzyl; 4-fluorobenzyl; 4-chlorophenylethyl; or benzyl.

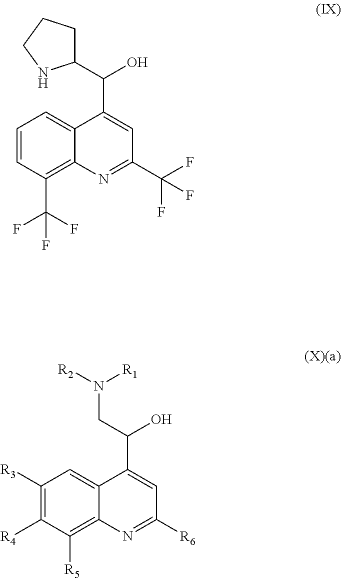

Where:

R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

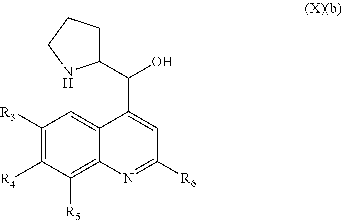

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; ethanone; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

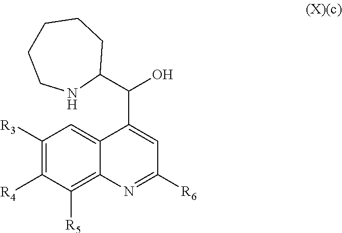

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

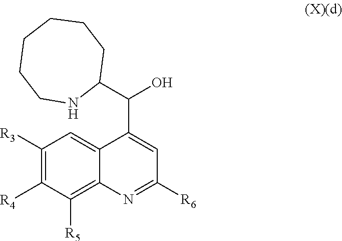

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

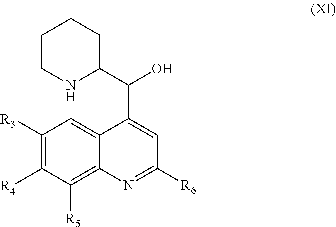

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

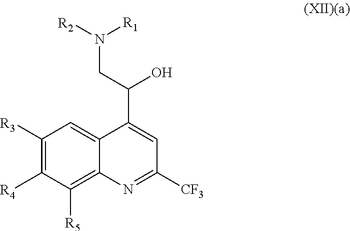

Where:

R₁ is methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

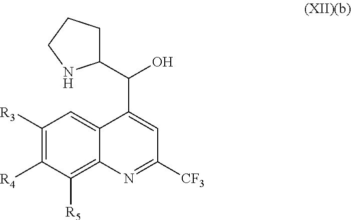

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

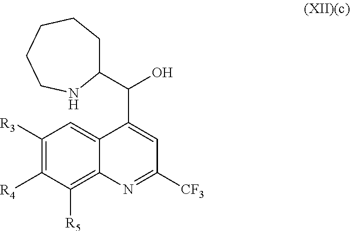

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

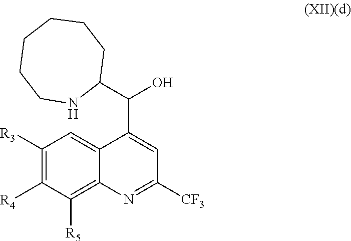

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

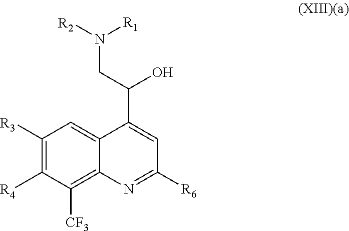

Where:

R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

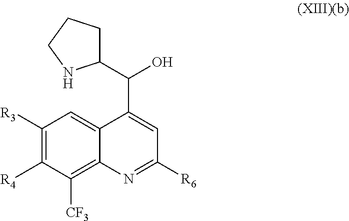

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

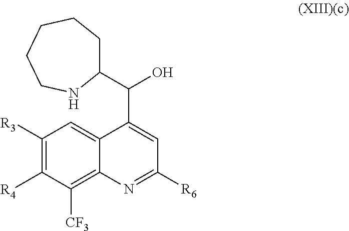

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

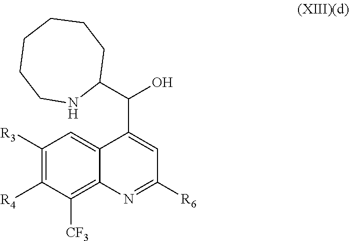

Where:

R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

Structures (VI)(a)-(c); (VII); (VIII); (IX), (X)(a)-(X)(d); (XI); (XII)(a)-(XII)(d); and (III)(a)-(XIII)(d), as shown and defined are useful for the method of treatment and/or prophylaxis of malaria, for the treatment of other infectious diseases, in particular those diseases caused by Microbacterium spp, including tuberculosis and leprosy for the treatment of parasitic diseases, in particular leishmaniasis and trypanosomiasis, diseases caused by other pathogenic nosocmial bacteria and fungi, for the treatment of autoimmune diseases including lupus, arthritis, sarcoidosis and rheumatism, for neurological conditions, including Parkinson's diseases and prion diseases including Creutxfeld-Jacob disease.

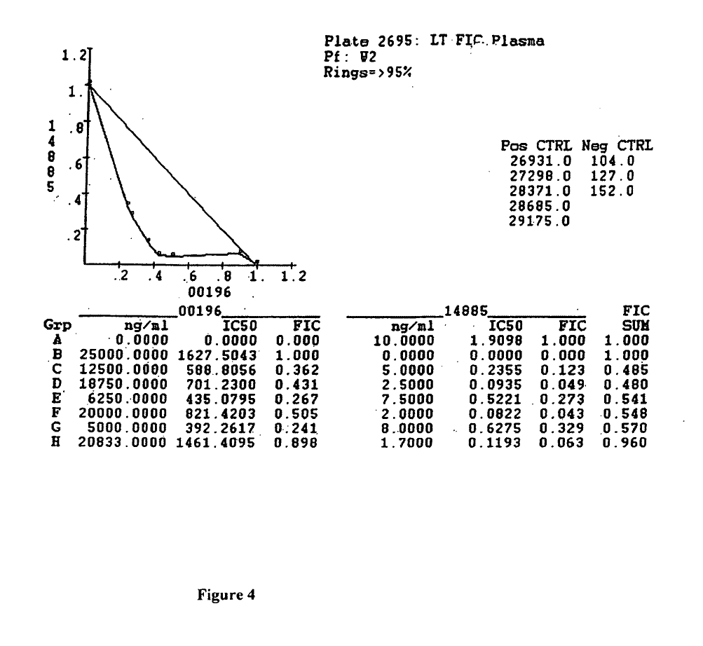

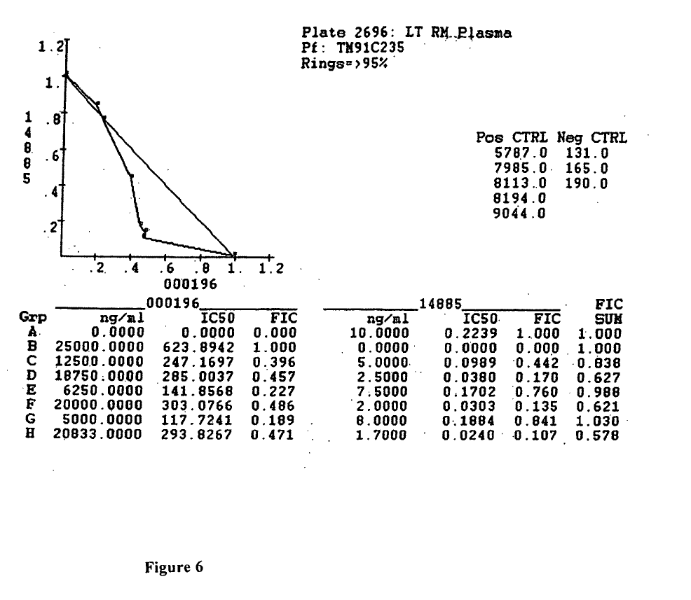

In accordance with the present invention, improvement of activity against infectious agents is also achieved by combination of AAQMs with azithromycin. The potential of AAQMS to act synergistically with azithromycin was evaluated utilizing isobologram analysis as described in the Materials and Methods. The interaction of azithromycin combined with WR074086 was investigated against P. falciparum D6, W2 and TM91C235. As can be seen in FIGS. 4 through 6, the line connecting the vertical (WR074086) and horizontal axis (azithromycin) is concave, below the line of additivity and the minimum fractional inhibitory concentration (FIC) is <0.5 in many instances. These data show that the combined effect of WR74086 and azithromycin against P. falciparum D6 is greater than the effect of either drug alone. Please refer to Materials and Methods for a description of the methods used. WR074086 is representative of the class of compound discussed herein Optimized mefloquine analog compounds exhibit greater potential as anti-infectives when used in combination with azithromycin than when used alone.

In accordance with present invention, next generation quinoline methanol compounds, as outlined in structures VI)(a)-(c); (VII); (VIII); (IX), (X)(a)-(X)(d); (XI); (XII)(a)-(XII)(d); and (III)(a)-(XIII)(d) above, can be combined with azithromycin for the treatment of infectious diseases, in particular malaria, tuberculosis, leishmania and trypanosomiasis. These quinoline methanol compounds are also be effective in combination with azithromycin against bacterial and fungal diseases. This is based on the observation that the activity of next generation quinoline methanols and azithromycin alone are less pronounced than when combined.

The following materials and methods are in accordance with the description of the embodiments of the present invention.

### In Vitro Antimalarial Activity of AAQMS.

The in vitro activities of AAQMs against P. falciparum strains W2, D6, TM91C235 and TM90C2A were evaluated using the method of Desjardins et al. as modified by Milhous et al. W2 is chloroquine resistant and mefloquine sensitive, D6 is chloroquine sensitive but naturally less susceptible to mefloquine, TM91C235 is resistant to mefloquine, chloroquine and pyrimethamine as is TM90C2A, however this latter parasite is a two pfmdr1 copy strain. We routinely run mefloquine in this screen as a control to ensure assay validity. Mefloquine has a mean IC50+/− SD against P. falciparum TM91C235 of 15.7+/−2.7 ng/ml (last 15 assays).

### Antimalarial Activity of AAQMs in Mice and Monkeys.

The P. berghei-mouse efficacy data were obtained from the WRAIR chemical information system (subcutaneous testing) or from recent tests conducted at AFRIMS, Thailand or at the University of Miami using a modified version of the Thompson test. Groups of five mice were inoculated through i.p. injection on day 0 (usually with 1×106 P. berghei-parasitized erythrocytes). The drugs in Table 1 were administered either subcutaneously or orally for three days (usually on days 3-5) at doses of 1.25-160 mg/kg in two to four fold increments. Cure was defined as survival until day 60 (subcutaneous dosing) or day 31 post-treatment (oral dosing). Nontreated control mice usually die on day 6-10 post-infection. The minimum effective dose was the lowest dose level that cured at least one of five mice. The Aotus studies were performed according to Obaldia; Detection of Klebsiella pnuumoniae antibodies in Aotus l. lemurinus (Panamanian owl monkey) using an enzyme linked immunosorbent assay (ELIA) test; Lab Anim (25)133-141 (1991) and Obaldia et al., WR 238605, chloroquine, and their combinations as blood schizonticides against a chloroquine-resistant strain of Plasmodium vivax in Aotus monkeys; Am. J. Trop. Med. Hyg. (56)508-510 (1997), which are incorporated by reference, herein. Groups of two Aotus lemurinus 6 lemurinus of the karyotype VIII or IX (27) male and female monkeys with weights ranging from 742-970 g were inoculated with 5×106 parasites of either the FVO strain of P. falciparum or the AMRU1 strain of P. vivax. Both of these strains are chloroquine resistant. The course of infection with P. falciparum is usually lethal, whilst the AMRU1 P. vivax strain induces a potentially lethal thrombocytopenia if left untreated. Monkeys were examined and thick Giemsa-stained blood smears were prepared and enumerated daily (17) to monitor the course of infection. When parasitemias increased to greater than 5000 parasites per μl, monkeys were treated orally with the test drug (those indicated in Table 1). If drug treatment failed, i.e. parasitemia did not decline, or increased again to >5000 parasites per μl, the monkeys were rescued with a single dose of orally administered mefloquine (20 mg/kg). In some instances, a high dose of the test drug was used to retreat monkeys instead of the mefloquine rescue. Monkeys are considered cured if they are parasite free 90 days post treatment. For the P. falciparum studies, the dose rate selected was 10 mg/kg/day×7, since earlier studies suggested that WR030090, a compound that later proceeded to clinical studies, cleared but did not cure infections at this dose (WRAIR archival data). Cure at this dose for the related analogs indicated in Table 1 would therefore be a good indicator of their superiority over a compound that had already proceeded into clinical development. Plasma samples were taken from the monkeys in some studies for quantification of metabolites. Additional blood samples were taken as appropriate for complete blood counts and serum chemistry. Research was conducted in compliance with the Animal Welfare Act and other federal statutes and regulations relating to animals and experiments involving animals and adheres to principles stated in the Guide for the Care and Use of Laboratory Animals, NRC Publication, 1996 edition (34).

### Metabolic Stability Analysis and Metabolite Determination.

The AAQMs indicated in Table 1 (10 μM) were added to a mixture containing an NADPH-regenerating buffer (1.25 mM β-NADP+, 3.3 mM glucose-6-phosphate, and 3.3 mM MgCl2) and 0.5 mg/ml pooled human liver microsomes to a final volume of 125 μl. The mixtures were incubated for 5 minutes at 37° C. and the reactions were initiated by adding 25 μl glucose-6-phosphate dehydrogenase to a final concentration of 1 unit/ml. The reactions were maintained at 37° C. until they were terminated by the addition of an equal volume of 100% ice cold acetonitrile at 0, 10, 30, 60, and 120 minutes. Samples were centrifuged to pellet the proteins and the supernatant was analyzed by LC-MS/MS in duplicate using fast LC gradient or isocratic methods. AAQM concentrations were quantified with external calibration, using plots of response v. amount. Chromatograms were analyzed using the mass spectrometry software Xcalibur® QuanBrowser (for ThermoFinnigan® instruments) or MassLynx® (for Waters® instruments). Concentrations of AAQMs remaining at each time point were calculated using the unknown peak areas and corresponding calibration curves. In order to calculate the half-life, a first-order rate of decay was assumed. The positive control tested with AAQM was nifedipine, which exhibited a mean half-life+/−SD of 31.7+/−5.3 min with human liver microsomes and 27.6+/−2.6 min with mouse liver microsomes (based on 5 assays). Mefloquine, which has been run >5 times in this assay, consistently exhibits a half-life of >120 min in the presence of both human and mouse 8 microsomes. All reagents were purchased from Sigma except for the microsomes, which were obtained from BD Gentest®. For metabolite identification, samples were prepared as described above with human liver microsomes. Additional samples were prepared with for each AAQM using mouse, rat, and rhesus monkey liver microsomes. Samples were separated using an LC gradient method and analyzed by full scan LC-MS and LC-MS/MS. AAQMs and putative metabolites were all fragmented, and these MS/MS experiments were used in combination with the no-NADPH control experiments to confirm the assignment of peaks as metabolites. These MS/MS data were also used to do preliminary structural elucidation. Although the dealkylated metabolites for each AAQMs were identified, the regioselectivity of other modifications, specifically the position(s) of hydroxylation, could not be definitively ascertained. The relative percentage of formation of each metabolite was determined in a semi-quantitative manner since standards of each metabolite were not available. Peak areas of each detected AAQM metabolite and the internal standard were determined, and their ratios were calculated as metabolite area/internal standard area. The percent formation of each metabolite was determined as the area ratio divided by the sum of all the metabolite area ratios.

### Neurocytotoxicity, Neuroprotection Assays and Confocal Microscopy.

The neurocytotoxicity assay of AAQMs was conducted as per Dow et al.; The antimalarial potential of 4-quinolinecarbinolamines may be limited due to neurotoxicity and cross-resistance in mefloquine-resistant Plasmodium falciparum strains; Chemother. (48) 2624-2632 (2004) and is incorporated herein, by reference. This assay utilizes primary rat forebrain neurons and is a multi-endpoint screen. In this system, a large component of the neurotoxicity of mefloquine is attributed to the disruption of calcium homeostasis via discharge of the endoplasmic reticulum calcium store and activation of ill-defined plasma membrane calcium channels. Mefloquine induces other, uncharacterized effects in these cells. The AAQMs outlined in Table 1 were screened at 10, 100 and 1000 μM in triplicate, and the reduction in viability observed was determined using the 3-(4,5-dimethylthiazol-2-yl)-2,5-diphenyltetrazolium bromide (MTT) assay as per Dow et al., discussed above. Approximate IC50 values were calculated on the basis of the level of inhibition observed at each concentration. Full dose response assays were run for mefloquine and WR069878 with two fold dilutions (n=6 wells per dilution) and 11 dilutions for each drug. IC50s were calculated using Prism. Each time the assay is conducted, 20 μM mefloquine is included, as a control. This concentration of mefloquine reduces cellular viability by a mean+/−SD of 52+/−4.4%. Assays are re-run if the loss of viability induced by mefloquine is <40% or >60%. Neuroprotection experiments with 6,7-dinitroquinoxaline-2,3-dione (DNQX) and magnesium were performed as per Dow et al.; Transcriptional profiling of mefloquine-induced disruption of calcium homeostasis in neurons in vitro; Genomics (86)539-550 (2005), which is incorporated by reference, herein. Neurons were exposed to DNQX (100 μM) or magnesium (12 mM) for 5 min, followed by mefloquine (25 μM) or WR069878 (250 μM) for 20 min after which reduction in cell viability was determined. Neither DNQX nor magnesium altered cellular viability alone. Each combination of treatments (mefloquine or WR069878 combined with DNQX or magnesium) was tested in quadruplicate on two occasions and similar trends were observed each time. DNQX is an inhibitor of non-N-methyl-D-aspartate (NMDA) receptors, whilst magnesium inhibits the functioning of the inositol 1,4,5-trisphosphate (IP3)-mediated calcium signaling pathway at several points. The effects of mefloquine and WR069878 on neuronal calcium homeostasis were assessed utilizing confocal microscopy as per Dow et al, as discussed in Antimicrob Agents Chemother. (48)2624-2632 (2004), cited above, and incorporated by reference, herein. Briefly neurons were loaded with calcium sensitive dye Fluo 3-A for 1 h and were washed prior to image experiments. Neurons were ‘spiked’ with 100 μM mefloquine or WR069878. Subsequent changes in neuronal calcium homeostasis were recorded as fluctuations in the emitted fluorescence of fluo-3-complexed calcium at 530 nm (excitation was 488 nm). Sequential image scans of fields containing 5-25 neurons were used to construct temporal profiles. Scans were made at 10 s intervals. Fluorescence levels for each neuron were normalized 12 to time zero values. Data were then pooled from three and five independent experiments for mefloquine and WR069878 respectively.

### Phototoxicity Pharmacophore and 3T3 Neutral Red Uptake Phototoxicity Test.

A pharmacophore for phototoxicity was developed to be used as an in silico screening tool to determine the minimum phototoxic concentration for a number of AAQMS (WR069878 and those indicated in Table 5). Minimum phototoxic concentration in a yeast assay for a number of AAQMS, as per Ison et al.; Phototoxicity of Quinoline methanols and other drugs in mice and yeast; J. Invest. Dermatol. (52)193-198 (1969) were used to generate a three-dimensional pharmacophore model using the HypoGen algorithm of the CATALYST® (Accelrys software Inc.) methodology as per Bhattacharjee et al.; A 3D QSAR pharmacophore model and quantum chemical structure—activity analysis of chloroquine(CQ)-resistance reversal; J. Chem Inf. Comput Sci. (42) 1212-1220 (2002), and Dow et al.; as discussed in Antimicrob Agents Chemother. (48)2624-2632 (2004), cited above, both Bhattacharjee et al. and Dow et al, incorporated herein, by reference. The structures of clinically used quinoline methanols were mapped onto the pharmacophores and estimated values for minimum phototoxic concentration were generated. The 3T3 neutral red uptake (NRU) phototoxicity test, conducted by MB Research Laboratories (Spinnerstown, Pa.), was used to identify quinoline methanols (WR069878 and those in Table 5) that have the potential to exert in vivo phototoxicity after systemic application. Briefly, the central 60 wells of two 96-well plates per AAQM were seeded with Balb/c 3T3 mouse fibroblast cells and maintained in culture for 24 hrs. These plates were then pre-incubated with a range of eight different concentrations of test compound (six wells per concentration) for one hour. Next, one plate was treated with a UVA dose of 5 J/cm2 by irradiating for 50 min at 1.7 mW/cm2, whereas the other plate remained non-treated and in the dark. Next, the treatment medium was replaced with culture medium and cell viability was determined after 24 hrs by measuring neutral red uptake for 3 hrs. Finally, the 3T3 NRU Phototox Prediction Software (version 2.0, ZEBET) was used to calculate EC50 values and Photo-Irritant Factors (PIF) for each compound. Compounds showing potential for phototoxicity have a PIF >5.0.

### Physiochemical Properties.

Important physiochemical properties including LogP, LogD, predicted solubility, polar surface area, pKA, number of H donors and acceptors, etc. were calculated using Advanced Chemistry Development LogD Sol Suite.

### Interaction Between Azithromycin and Quinoline Methanols:

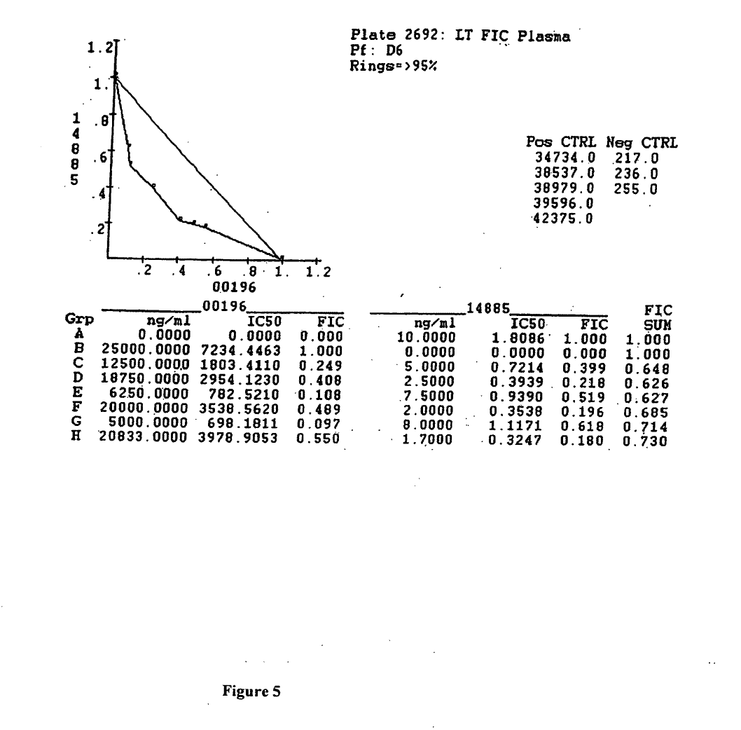

The combined effects of azithromycin (as represented by compound 196 on the horizontal axes of FIG. 4 through 6) and WR074086 (as represented by compound 14885 on vertical axes of FIGS. 4 through 6) against three strains of P. falciparum were investigated using isobologram analysis. First, the IC50s of each compound were determined as described above. These are represented on the first two lines of the data table on each of FIGS. 4-6. For example, for P. falciparum D6 (FIG. 5), the IC50 for azithromycin was 7234 ng/ml, and the highest concentration tested was 25000 ng/ml (line B), whereas for WR074086 the IC50 was 1.8086 ng/ml with a starting concentration of 10 ng/ml (line A).

Next, IC50s were determined with different fractional starting concentrations of each compound. For example in line C of FIG. 5, the starting concentration of azithromycin was 12500 ng/ml whereas the starting concentration of WR074086 was 5 ng/ml. Each of these starting concentrations is half of that used when each compound was tested alone. Although it is expected if the two compounds were additive, their IC50s in combination would be half that of each of the compound alone, lower than expected IC50s were observed. In the data tables, lines D-H represent other combinations of starting concentrations of each compound.

For each pair of starting concentrations for each drug, fractional inhibitory concentrations were determined using the following formula: FIC=IC50 in combination/IC50 alone. For each pair, the FIC sum was calculated using the following formula: FIC SUM=FIC DRUG A+FIC DRUG B. For example, in Line C on FIG. 5, the FIC of azithromycin is 0.249, the FIC of WR074086 is 0.399, and the FIC sum is 0.648.

The FICs for the eight tests are plotted as indicated in FIG. 4 through FIG. 6 for each of three P. falciparum strains. Graphs of this type are called isobolograms and additivity is represented by an overall FIC SUM of 1, as represented by the straight line joining the two axes. A potentiative effect is represented by a concave curve below the straight line, since IC50s in combination are lower than expected. Antagonism is represented by a convex curve above the straight line, since IC50s in combination are higher than expected.

What is claimed is:

1. A method for treating and preventing an individual with a parasitic infection comprising administering to said individual an improved quinoline analog compounds in a pharmaceutically effective amount, in a pharmaceutically effective excipient.

2. A method as recited in claim 1, wherein said administration is selected from the group consisting of oral, topical, transdermal and parenteral.

3. A method as recited in claim 2, wherein said individual is a human.

4. A method as recited in claim 2, wherein said individual is an animal.

5. A method as recited in claim 3 or 4 wherein the parasitic infection is malaria.

6. A method as recited in claim 5 wherein said malaria stains are selected from the group consisting of P. falciparum, P. berghei, P. vivax and TM90C2A, TM91C235, D6 and W2.

7. A method as recited in claim 3 or 4 wherein said parasitic infection is selected from a group consisting of tuberculosis, trypanomiasis and leishamaniasis.

8. A method as recited in claim 6, wherein said improved quinoline analog compounds are made from said quinoline analog compound made from 2-substituted alkylquinolinyl methanols having the structure: 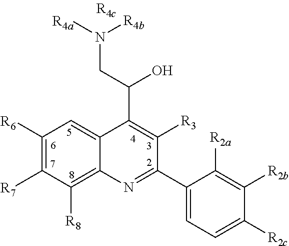
where: R₂ₐ is H or t-butyl; R_{2b} is H or Cl; R_{2c} is H, Cl, or F; R₃ is H; R₄ₐ is H, ethyl, butyl or hexyl; R_{4b} is H, ethyl, butyl, or hexyl; R_{4c} represents an addition to the N or the amino side chain; R₆ is H, methyl or Cl; R₇ is H, F, or Cl; and R₈ is H, methyl or Cl.

9. A method as recited in claim 8 wherein said improved quinoline compound is: 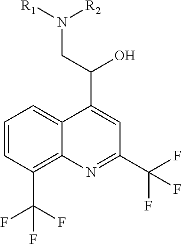
Where:
R₁ is Me and R₂ is H; R₁ and R₂ are propyl groups; R₁ is H and R₂ is a propyl group; R₁ is H and R₂ is CH₂CHOH—CH₂—CH₃; R₁ is H and R₂ is CH₂—CH₂—CHOH—CH₃; R₁ is H, R₂ is CH₂—CH₂—CH₂—CH₂OH; R₁ is OH and R₂ is butyl; R₁ is a butyl group and R₂ is CH₂OH; R₁ is butyl and R₂ is CH₂—CH₂—COOH; R₁ is CH₂—CH₂—COOH and R₂ is CH₂—CH₂—COOH; R₁ is H and R₂ is CH₂—CH₂—COOH; or R₁ and R₂ are cyclopropyls.

10. A method as recited in claim 8 wherein said improved quinoline compound is:

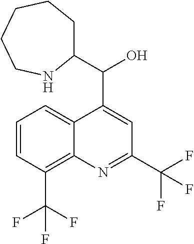

11. A method as recited in claim 8 wherein said improved quinoline compound is:

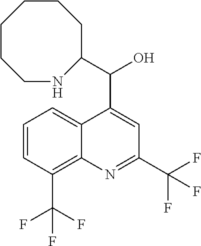

12. A method as recited in claim 8 wherein said improved quinoline compound is: 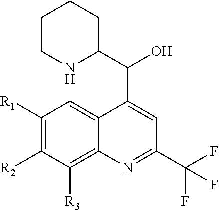
Where:
R₁ is Me, R₂ is Cl and R₃ is H; R₁ is Cl, R₂ is Me, R₃ is H; R₁ and R₂ are Me, R₃ is H; R₁ and R₂ are Cl and R₃ is H; R₁ is H, R₂ and R₃ are Cl; R₁ and R₂ are H and R₃ is a hydroxy group; R₁ and R₂ are H and R₃ is CH₂OH; R₁ and R₂ are H and R₃ is ethanone; R₁ and R₂ are H and R₃ is methylhydroxy; or R₁ and R₂ are H and R₃ is trifluoromethoxy.

13. A method as recited in claim 8 wherein said improved quinoline compound is: 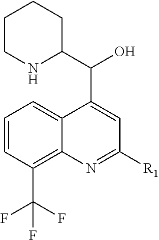
Where: R₁ is 2,6-dichlorophenyl; 2,6-dimethylphenyl; 2-6-bis(trifluoromethyl)phenyl; 4-chlorobenzyl; 4-fluorobenzyl; 4-chlorophenylethyl; or benzyl.

14. A method as recited in claim 8 wherein said improved quinoline compound is:

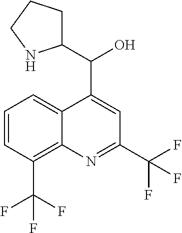

15. A method as recited in claim 8 wherein said improved quinoline compound is: 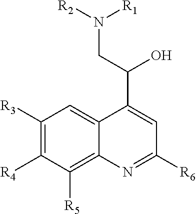
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

16. A method as recited in claim 8 wherein said improved quinoline compound is: 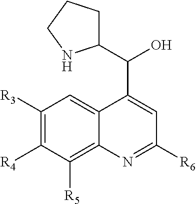
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; ethanone; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

17. A method as recited in claim 8 wherein said improved quinoline compound is: 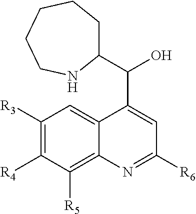
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

18. A method as recited in claim 8 wherein said improved quinoline compound is: 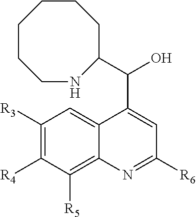
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

19. A method as recited in claim 8 wherein said improved quinoline compound is: 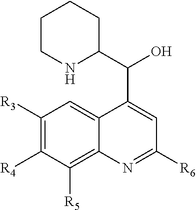
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

20. A method as recited in claim 8 wherein said improved quinoline compound is: 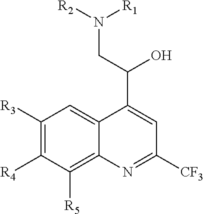
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

21. A method as recited in claim 8 wherein said improved quinoline compound is: 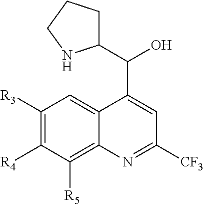
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

22. A method as recited in claim 8 wherein said improved quinoline compound is: 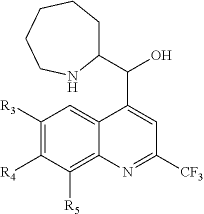
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

23. A method as recited in claim 8 wherein said improved quinoline compound is: 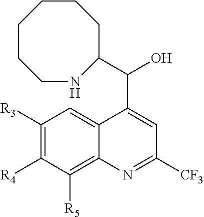
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

24. A method as recited in claim 8 wherein said improved quinoline compound is: 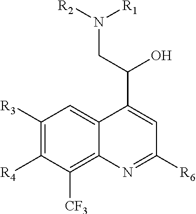
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

25. A method as recited in claim 8 wherein said improved quinoline compound is: 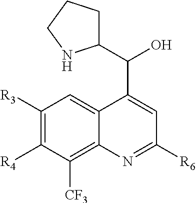
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

26. A method as recited in claim 8 wherein said improved quinoline compound is: 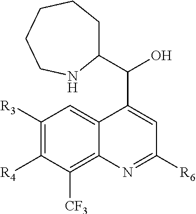
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

27. A method as recited in claim 8 wherein said improved quinoline compound is: 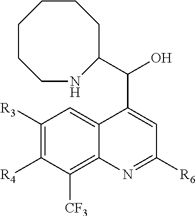
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

28. An antiparasitic compound comprising an antiparasitic effective amount of an improved quinoline analog compound having metabolic stability, reduced neurotoxicity and increased activity, said quinoline analog compound having a 2-substituted alkylquinolinyl methanols starting compound of the structure 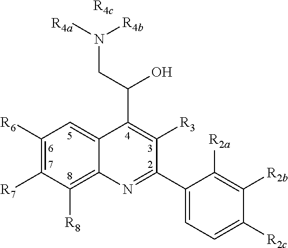
R₂ₐ is H or t-butyl; R_{2b} is H or Cl; R_{2c} is H, Cl, or F; R₃ is H; R₄ₐ is H, ethyl, butyl or hexyl; R_{4b} is H, ethyl, butyl, or hexyl; R_{4c}, represents an addition to the N or the amino side chain; R₆ is H, methyl or Cl; R₇ is H, F, or Cl; and R₈ is H, methyl or Cl.

29. An antiparasitic compound as recited in claim 28, wherein said improved quinoline analog compound is: 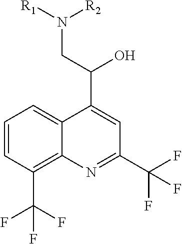
Where:
R₁ is Me and R₂ is H; R₁ and R₂ are propyl groups; R₁ is H and R₂ is a propyl group; R₁ is H and R₂ is CH₂CHOH—CH₂—CH₃; R₁ is H and R₂ is CH₂—CH₂—CHOH—CH₃; R₁ is H, R₂ is CH₂—CH₂—CH₂—CH₂OH; R₁ is OH and R₂ is butyl; R₁ is a butyl group and R₂ is CH₂OH; R₁ is butyl and R₂ is CH₂—CH₂—COOH; R₁ is CH₂—CH₂—COOH and R₂ is CH₂—CH₂—COOH; R₁ is H and R₂ is CH₂—CH₂—COOH; or R₁ and R₂ are cyclopropyls.

30. An antiparasitic compound as recited in claim 28, wherein said improved quinoline analog compound is:

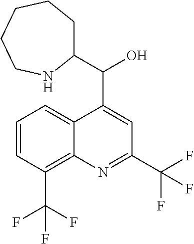

31. An antiparasitic compound as recited in claim 28 wherein said improved quinoline analog compound is:

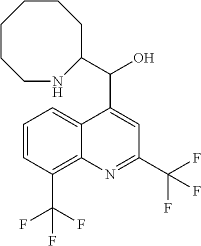

32. An antiparasitic compound as recited in claim 28, wherein said improved quinoline analog compound is: 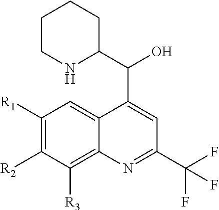
Where:
R₁ is Me, R₂ is Cl and R₃ is H; R₁ is Cl, R₂ is Me, R₃ is H; R₁ and R₂ are Me, R₃ is H; R₁ and R₂ are Cl and R₃ is H; R₁ is H, R₂ and R₃ are Cl; R₁ and R₂ are H and R₃ is a hydroxy group; R₁ and R₂ are H and R₃ is CH₂OH; R₁ and R₂ are H and R₃ is ethanone; R₁ and nd R₂ are H and R₃ is methylhydroxy; or R₁ and R₂ are H and R₃ is trifluoromethoxy.

33. An antiparasitic compound as recited in claim 28, wherein said improved quinoline analog compound is: 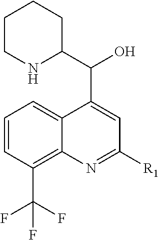
Where: R₁ is 2,6-dichlorophenyl; 2,6-dimethylphenyl; 2-6-bis(trifluoromethyl)phenyl; 4-chlorobenzyl; 4-fluorobenzyl; 4-chlorophenylethyl; or benzyl.

34. An antiparasitic compound as recited in claim 28, wherein said improved quinoline analog compound is:

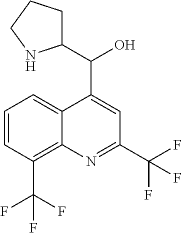

35. An antiparasitic compound as recited in claim 28, wherein said improved quinoline analog compound is: 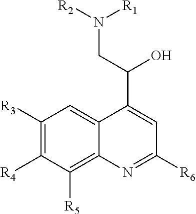
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

36. An antiparasitic compound as recited in claim 28, wherein said improved quinoline analog compound is: 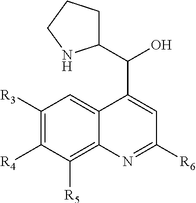
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; ethanone; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

37. An antiparasitic compound as recited in claim 28, wherein said improved quinoline analog compound is: 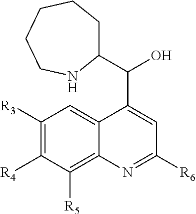
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

38. An antiparasitic compound as recited in claim 28, wherein said improved quinoline analog compound is: 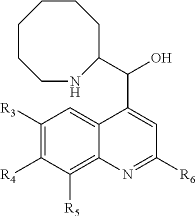
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

39. An antiparasitic compound as recited in claim 28, wherein said improved quinoline analog compound is: 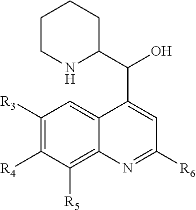
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

40. An antiparasitic compound as recited in claim 28, wherein said improved quinoline analog compound is: 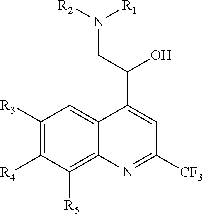
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

41. An antiparasitic compound as recited in claim 28, wherein said improved quinoline analog compound is: 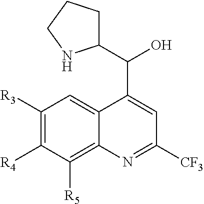
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

42. An antiparasitic compound as recited in claim 28, wherein said improved quinoline analog compound is: 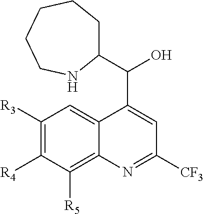
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

43. An antiparasitic compound as recited in claim 28, wherein said improved quinoline analog compound is: 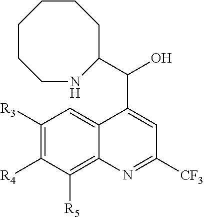
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

44. An antiparasitic compound as recited in claim 28, wherein said improved quinoline analog compound is: 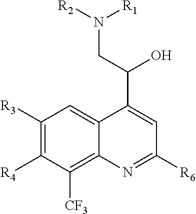
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

45. An antiparasitic compound as recited in claim 28, wherein said improved quinoline analog compound is: 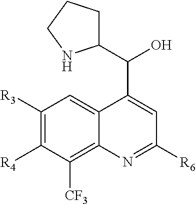
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

46. An antiparasitic compound as recited in claim 28, wherein said improved quinoline analog compound is: 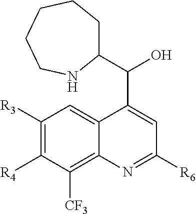
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

47. An antiparasitic compound as recited in claim 28, wherein said improved quinoline analog compound is: 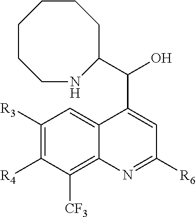
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

48. A method of making improved quinoline analog compounds comprising the steps of:
(i) selecting a 2-phenyl substituted dialkylquinolinyl methanols having a quinoline ring as starting compounds;
(ii) selecting a 4-side chain substituent that appropriately balances metabolic stability with reduced neurotoxicity and increased antiparasitic activity;
(iii) selecting a 2 position substituent that optimizes antiparasitic activity and reduces phototoxicity;
(iv) adding a substitute at 6, 7, and 8 position of the quinoline ring and optimizing activity and ensure ease of synthesis; and
(v) introducing additional moieties and improving oral absorption, reducing blood brain barrier passage, and decreasing PgP substrate affinity.

49. A method according to claim 48, wherein said starting compounds are: 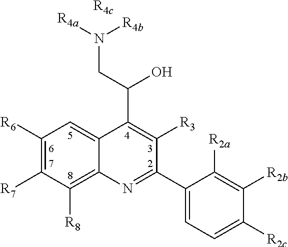
Where:
R₂ₐ is H or t-butyl; R_{2b} is H or Cl; R_{2c} is H, Cl, or F; R₃ is H; R₄ₐ is H, ethyl, butyl or hexyl; R_{4b} is H, ethyl, butyl, or hexyl; R_{4c} represents an addition to the N or the amino side chain; R₆ is H, methyl or Cl; R₇ is H, F, or Cl; and R₈ is H, methyl or Cl.

50. A method according to claim 49, wherein said step (ii) further comprises removing a side chain piperidine ring of said quinoline methanol compounds so as to increase activity.

51. A method according to claim 50, wherein said step (ii) comprises selecting substituents that are resistant to N-dealkylation, increases potency and has reduced neurotoxicity.

52. A method according to claim 51 wherein said step (ii) substituents are N-butyl(mono) side chain.

53. A method according to claim 52, wherein said step (ii) substituent is:

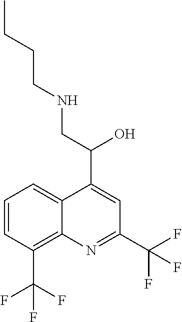

54. A method according to claim 52, wherein said step (ii) substituent is:

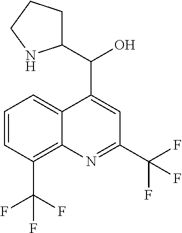

55. A method according to claim 52, wherein said step (ii) substituent is:

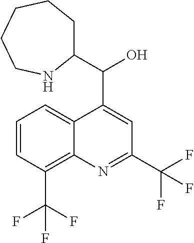

56. A method according to claim 52, wherein said step (ii) substituent is:

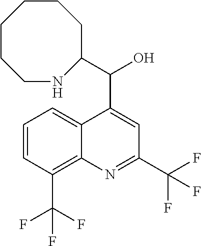

57. A method according to claim 53, 54, 55 or 56, wherein said step (iii) substituent further comprises:

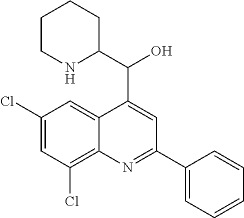

58. A method according to claim 53, 54, 55, or 56, wherein said step (iii) substituent further comprises:

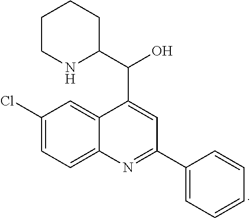

59. A method according to claim 53, 54, 55, or 56, wherein said step (iii) substituent further comprises:

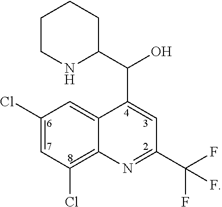

60. A method according to claim 53, 54, 55 or 56, wherein said step (iii) substituent further comprises:

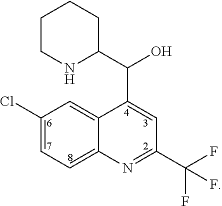

61. A method according to claim 53, 54, 55 or 56, wherein said step (iii) substituent further comprises:

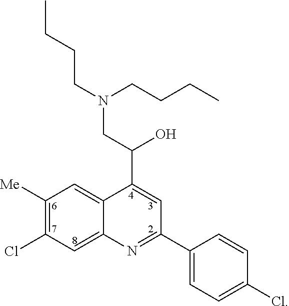

62. A method according to claim 53, 54, 55, or 56, wherein said step (iii) substituent further comprises:

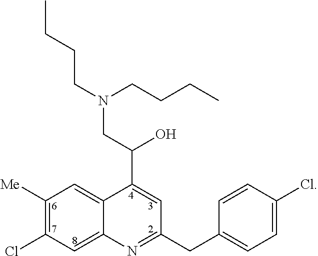

63. A method according to claim 53, 54, 55 or 56, wherein said step (iii) substituent further comprises:

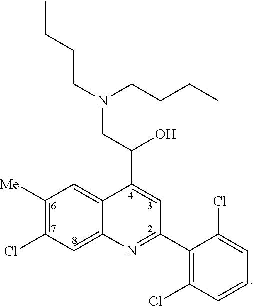

64. A method according to claim 52, wherein said step (v) substituent is:

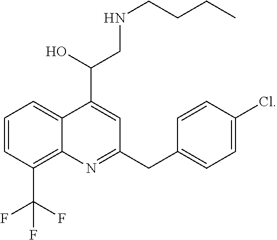

65. A method according to claim 52, wherein said step (v) substituent is:

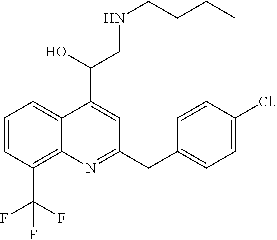

66. A method according to claim 52, wherein said step (v) substituent is:

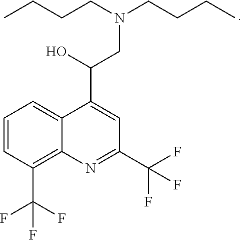

67. A method according to claim 52, wherein said step (v) substituent is:

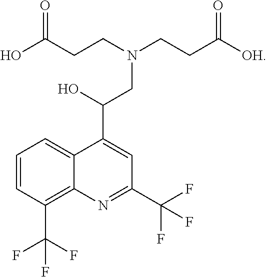

68. A method for treating and preventing parasitic infections in individuals comprising administering to said individual an improved quinoline analog compounds in combination with azithromycin, in a pharmaceutically effective amount, in a pharmaceutically effective excipient.

69. A method as recited in claim 68, wherein said administration is selected from the group consisting of oral, topical, transdermal and parenteral.

70. A method as recited in claim 69, wherein said individual is a human.

71. A method as recited in claim 69, wherein said individual is an animal.

72. A method as recited in claim 70 or 71, wherein the parasitic infection is malaria.

73. A method as recited in claim 72, wherein said malaria stains are selected from the group consisting of P. falciparum, P. berghei, P. vivax and TM90C2A, TM91C235, D6 and W2.

74. A method as recited in claim 70 or 71 wherein said parasitic infection is selected from a group consisting of tuberculosis, trypanomiasis and leishamaniasis.

75. A method as recited in claim 73, wherein said improved quinoline analog compounds are made from said quinoline analog compound made from 2-substituted alkylquinolinyl methanols having the structure: 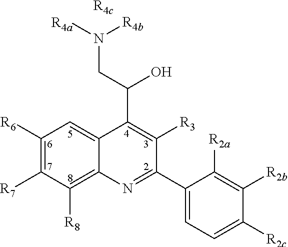
where:
R₂ₐ is H or t-butyl; R_{2b} is H or Cl; R_{2c} is H, Cl, or F; R₃ is H; R₄ₐ is H, ethyl, butyl or hexyl; R_{4b} is H, ethyl, butyl, or hexyl; R_{4c} represents an addition to the N or the amino side chain; R₆ is H, methyl or Cl; R₇ is H, F, or Cl; and R₈ is H, methyl or Cl.

76. A method as recited in claim 75 wherein said improved quinoline compound is: 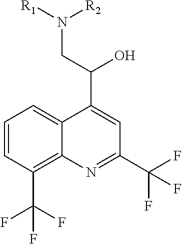
Where:
R₁ is Me and R₂ is H; R₁ and R₂ are propyl groups; R₁ is H and R₂ is a propyl group; R₁ is H and R₂ is CH₂CHOH—CH₂—CH₃; R₁ is H and R₂ is CH₂—CH₂—CHOH—CH₃; R₁ is H, R₂ is CH₂—CH₂—CH₂—CH₂OH; R₁ is OH and R₂ is butyl; R₁ is a butyl group and R₂ is CH₂OH; R₁ is butyl and R₂ is CH₂—CH₂—COOH; R₁ is CH₂—CH₂—COOH and R₂ is CH₂—CH₂—COOH; R₁ is H and R₂ is CH₂—CH₂—COOH; or R₁ and R₂ are cyclopropyls.

77. A method as recited in claim 75 wherein said improved quinoline compound is:

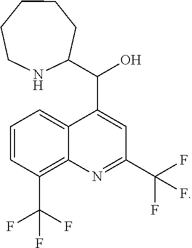

78. A method as recited in claim 75 wherein said improved quinoline compound is:

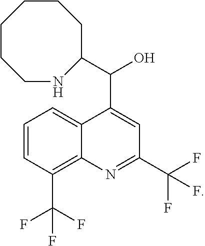

79. A method as recited in claim 75 wherein said improved quinoline compound is: 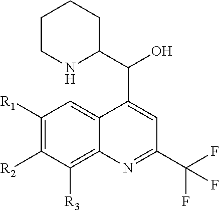
Where:
R₁ is Me, R₂ is Cl and R₃ is H; R₁ is Cl, R₂ is Me, R₃ is H; R₁ and R₂ are Me, R₃ is H; R₁ and R₂ are Cl and R₃ is H; R₁ is H, R₂ and R₃ are Cl; R₁ and R₂ are H and R₃ is a hydroxy group; R₁ and R₂ are H and R₃ is CH₂OH; R₁ and R₂ are H and R₃ is ethanone; R₁ and R₂ are H and R₃ is methylhydroxy; or R₁ and R₂ are H and R₃ is trifluoromethoxy.

80. A method as recited in claim 75 wherein said improved quinoline compound is: 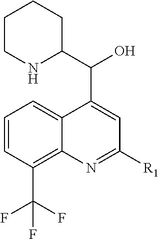
Where: R₁ is 2,6-dichlorophenyl; 2,6-dimethylphenyl; 2-6-bis(trifluoromethyl)phenyl; 4-chlorobenzyl; 4-fluorobenzyl; 4-chlorophenylethyl; or benzyl.

81. A method as recited in claim 75 wherein said improved quinoline compound is:

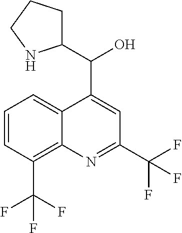

82. A method as recited in claim 75 wherein said improved quinoline compound is: 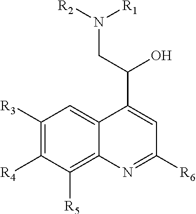
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

83. A method as recited in claim 75 wherein said improved quinoline compound is: 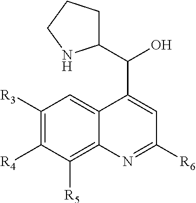
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; ethanone; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

84. A method as recited in claim 75 wherein said improved quinoline compound is: 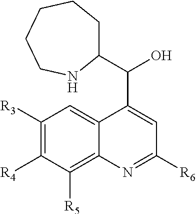
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

85. A method as recited in claim 75 wherein said improved quinoline compound is: 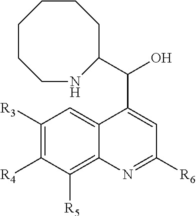
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

86. A method as recited in claim 75 wherein said improved quinoline compound is: 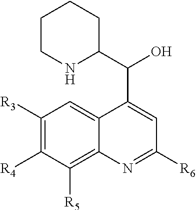
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

87. A method as recited in claim 75 wherein said improved quinoline compound is: 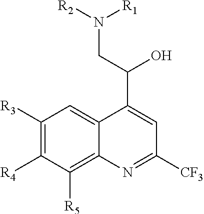
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

88. A method as recited in claim 75 wherein said improved quinoline compound is: 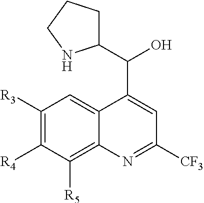
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

89. A method as recited in claim 75 wherein said improved quinoline compound is: 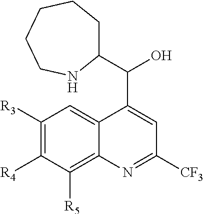
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

90. A method as recited in claim 75 wherein said improved quinoline compound is: 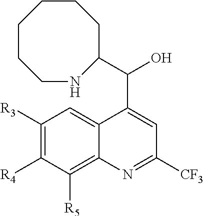
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH;
R₅ is H; methoxy; methyl; chloro; hydroxyl; ethanone; methylhydroxy; trifluoromethoxy; or CH₂OH.

91. A method as recited in claim 75 wherein said improved quinoline compound is: 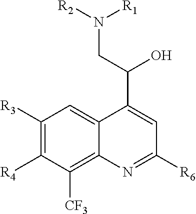
Where:
R₁ is H, methyl, ethyl, propyl, butyl, hydroxy, cyclopropyl, CH₂—CHOH—CH2-CH₃; CH₂—CH₂—CHOH—CH₃; CH₂—CH₂—CH₂—CH₂OH; CH₂OH; or CH₂—CH₂—COOH;
R₂ is H; methyl; ethyl; propyl; butyl; hydroxyl; cyclopropyl; CH₂—CHOH—CH₂—CH₃; CH₂—CH₂—CHOH—CH₃; CH₂—CH₂—CH₂—CH₂OH; CH₂OH; or CH₂—CH₂—COOH;
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

92. A method as recited in claim 75 wherein said improved quinoline compound is: 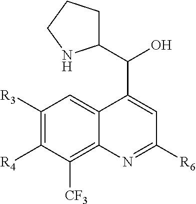
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

93. A method as recited in claim 75 wherein said improved quinoline compound is: 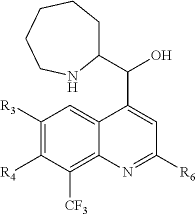
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.

94. A method as recited in claim 75 wherein said improved quinoline compound is: 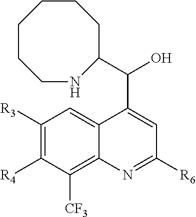
Where:
R₃ is H; trifluoromethyl; methoxy; methyl; chloro; hydroxyl; ethanone; methylhydroxy; trifluoromethoxy; or CH₂OH;
R₄ is H; trifluoromethyl; methoxy; methyl; chloro; hydroxyl; ethanone; methylhydroxy; trifluoromethoxy; or CH₂OH; and
R₆ is 2,6-dichlorophenyl; 2,6-dimethylphenyl; 2,6-bis(trifluoromethy)phenyl; 4-chlorobenzyl; 4-fluorobenzyl; 4-chlorophenylethyl; or benzyl.
